(19) United States
(12) Patent Application Publication
(10) Pub. No.: US 2017/0128518 A1
(43) Pub. Date: May 11, 2017

(54) METHODS AND MATERIALS FOR IDENTIFYING AND TREATING MAMMALS RESISTANT TO PROTEASOME INHIBITOR TREATMENTS

(71) Applicant: Mayo Foundation for Medical Education and Research, Rochester, MN (US)

(72) Inventors: Kena C. Miller, Jacksonville, FL (US); Aneel Paulus, Jacksonville, FL (US); Kasyapa Chitta, Jacksonville, FL (US); Asher Alban Akmal Chanan-Khan, Ponte Vedra Beach, FL (US)

(73) Assignee: Mayo Foundation for Medical Education and Research, Rochester, MN (US)

(21) Appl. No.: 15/322,614

(22) PCT Filed: Jul. 1, 2015
(86) PCT No.: PCT/US15/38859

§ 371 Date: Dec. 28, 2016

Related U.S. Application Data
(60) Provisional application No. 62/019,646, filed on Jul. 1, 2014.

(51) Int. Cl. A61K 38/05; A61K 31/55; A61K 38/07; C12Q 1/68; G01N 33/574

(52) U.S. Cl. CPC A61K 38/05; C12Q 1/6886; G01N 33/57407; A61K 38/07; A61K 31/55; C12Q 2600/158; G01N 2333/47

(57) ABSTRACT

This document provides methods and materials involved in identifying mammals having blood cancer (e.g., myelomas or lymphomas, including WM, MCL, and DLBCL) that is resistant to treatment with a proteasome inhibitor such as bortezomib (e.g., VELCADE®) as well as methods and materials involved in treating mammals having a blood cancer resistant to a proteasome inhibitor such as bortezomib (e.g., VELCADE®). For example, methods and materials for using the expression level of PSMB9/β1i nucleic acid to identify a mammal as having a blood cancer (e.g., myeloma or lymphoma, such as WM, MCL, or DLBCL) that is resistant to treatment with a proteasome inhibitor such as bortezomib (e.g., VELCADE®) are provided.

### CROSS-REFERENCE TO RELATED APPLICATIONS

This application claims benefit of priority from U.S. Provisional Application No. 62/019,646, filed on Jul. 1, 2014.

### BACKGROUND

1. Technical Field

This document relates to methods and materials involved in identifying mammals having blood cancer (e.g., lymphomas or myelomas) resistant to treatment with particular proteasome inhibitors (e.g., bortezomib (e.g., VELCADE®)) as well as methods and materials involved in treating mammals having a blood cancer resistant to particular proteasome inhibitors. For example, this document provides methods and materials for using the expression level of proteasome subunit beta type-9 (PSMB9) nucleic acid, which encodes a β1i subunit, to identify a mammal as having a blood cancer (e.g., lymphomas or myelomas) resistant to treatment with a proteasome inhibitor.

2. Background Information

In the United States, over one million people are estimated to be living with, or in remission from, blood cancers. Malignant B-lymphocyte neoplasms are the second most common form of hematologic cancer and despite impressive progress in new therapeutic development, they remain incurable. Blood cancers encompassing multiple myeloma (MM) and certain Non-Hodgkin's lymphomas (NHL) such as mantle cell lymphoma (MCL), plasmacytic lymphoma (also known as Waldenströms macroglobulinemia; WM), follicular lymphoma (FL), and diffuse large B-cell lymphoma (DLBCL) have been found to rely on optimal performance of the ubiquitin-proteasomal degradation system (UPS). In comparison to normal, the malignantly transformed lymphocytes have a significantly higher protein turnover rate and therefore are highly sensitive to proteasome inhibitors (PI) such as bortezomib (e.g., VELCADE®) or carfilzomib (e.g., KYPROLIS®).

Patients with the maladies mentioned above derive a significant clinical benefit from treatment with bortezomib-based therapies, exhibiting response rates between about 100% and about 75% in treatment naïve MM patients (Reeder et al., Blood, 115(16):3416-3417, 2010; and Richardson et al., Blood, 116(5):679-686, 2010) and MCL (Orciuolo et al., Br. J. Haematol., 148(5):810-812, 2010; and Friedberg et al., Blood, 117(10):2807-2812, 2011) patients, respectively. Comparable efficacy of bortezomib-containing regimens was confirmed in all subtypes of NHL (Fowler et al., J. Clin. Oncol., 29(25):3389-3395, 2011; and Boswell et al., Blood, 122(21):4402, 2013). Indeed, the proteasome and the UPS comprise essential components of normal cellular homeostasis and are critical to malignant B-cell survival. Despite high response rates, all patients, however, acquire resistance to bortezomib or carfilzomib, and this is associated with a highly aggressive disease phenotype (Ruschak et al., J. Nat. Cancer Inst., 103(13):1007-1017, 2011).

### SUMMARY

This document provides methods and materials involved in identifying mammals having blood cancer (e.g., myeloma or lymphoma, including WM, MCL, and DLBCL) resistant to treatment with a proteasome inhibitor such as bortezomib (e.g., VELCADE®) as well as methods and materials involved in treating mammals having a blood cancer resistant to a proteasome inhibitor such as bortezomib (e.g., VELCADE®). For example, this document provides methods and materials for using the expression level of PSMB9 nucleic acid to identify a mammal as having a blood cancer (e.g., myeloma or lymphoma, such as WM, MCL, or DLBCL) resistant to treatment with a proteasome inhibitor such as bortezomib (e.g., VELCADE®). As described herein, the presence of an elevated level of expression of PSMB9 nucleic acid or an elevated level of β1i polypeptides within blood cancer cells (e.g., myeloma or lymphoma cells, including WM, MCL, or DLBCL cells) from a mammal can indicate that that mammal (e.g., a human) has a blood cancer resistant to a proteasome inhibitor that targets the β5 subunit of a proteasome such as bortezomib. As also described herein, a mammal with a blood cancer (e.g., myeloma or lymphoma, such as WM, MCL, or DLBCL) can be treated by detecting the presence of an elevated level of expression of PSMB9 nucleic acid within blood cancer cells (e.g., myeloma or lymphoma cells, such as WM, MCL, or DLBCL cells) or an elevated level of β1i polypeptides within blood cancer cells and administering carfilzomib (e.g., KYPROLIS®) or other drugs that can potentially bypass the β1i and β5 (such as VLX1570) to that mammal. While not being limited to any particular mode of action, the use of carfilzomib (e.g., KYPROLIS®) appears to kill blood cancer cells in a manner independent of PSMB-5 inhibition (Sacco et al., Clin. Cancer Res., 17(7):1753-64 (2011)).

Having the ability to identify mammals as having a blood cancer resistant to a proteasome inhibitor that targets the β5 subunit of a proteasome such as bortezomib as described herein can allow those blood cancer patients to be properly identified and treated in an effective and reliable manner with either bortezomib-based therapy (if PSMB9 is present in a decreased amount) or with non-bortezomib-based therapies. For example, the blood cancer treatments provided herein (e.g., carfilzomib or VLX1570) can be used to treat blood cancer patients identified as having blood cancer resistant to a proteasome inhibitor that targets the β5 subunit of a proteasome such as bortezomib.

In general, one aspect of this document features a method for identifying a mammal as having blood cancer cells resistant or susceptible to treatment with bortezomib. The method comprises, or consists essentially of, (a) detecting the presence or absence of blood cancer cells having an elevated level of PSMB9 nucleic acid expression in the mammal, wherein the mammal received treatment with bortezomib, and (b) classifying the mammal as having blood cancer cells resistant to treatment with bortezomib if the presence of the blood cancer cells is detected, and classifying the mammal as having blood cancer cells susceptible to treatment with bortezomib if the absence of the blood cancer cells is detected. The mammal can be a human. The presence or absence of the blood cancer cells can be detected using a quantitative polymerase chain reaction assay to measure PSMB9 mRNA levels. The blood cancer cells can be lymphoma cells (e.g., WM, FL, MCL, or DLBCL cells). The blood cancer cells can be myeloma cells. The presence can be detected, and the mammal can be classified as having blood cancer cells resistant to treatment with bortezomib. The absence can be detected, and the mammal can be classified as having blood cancer cells susceptible to treatment with bortezomib.

In another aspect, this document features a method for treating blood cell cancer in a mammal. The method can comprise, or consist essentially of: (a) administering bortezomib to said mammal, (b) detecting the presence of blood cancer cells within said mammal that have an elevated level of PSMB9 expression, and (c) administering carfilzomib to said mammal. The mammal can be a human. The presence of said blood cancer cells can be detected using a quantitative polymerase chain reaction assay to measure PSMB9 mRNA levels, or can be detected using a polypeptide detection assay for detecting β1i polypeptide levels. The blood cancer cells can be lymphoma cells (e.g., mantle cell lymphoma (MCL) cells, Waldenstroms macroglobulinemia (WM) cells, or diffuse large B-cell lymphoma (DLBCL) cells). The blood cancer cells can be myeloma cells.

In another aspect, this document features a method for treating blood cell cancer in a mammal, where the method comprises or consists essentially of: (a) administering bortezomib to said mammal, (b) detecting the presence of blood cancer cells within said mammal that have an elevated level of PSMB9 expression, and (c) administering VLX1570 to said mammal. The mammal can be a human. The presence of said blood cancer cells can be detected using a quantitative polymerase chain reaction assay to measure PSMB9 mRNA levels, or can be detected using a polypeptide detection assay for detecting β1i polypeptide levels. The blood cancer cells can be lymphoma cells (e.g., MCL cells, WM cells, or DLBCL cells). The blood cancer cells can be myeloma cells.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C show the characterization of bortezomib-resistance (BR) models, established by subjecting plasma cell cancer cell lines (n=6) chronic drug exposure. FIG. 1A is a graph plotting the results of an MTS assay for an OPM2 cell line (representative model). The IC₅₀ of the WT (wild type) was 6.14 nM, while the IC₅₀ for the corresponding BR derivative was >50 nM, indicating complete insensitivity to bortezomib. FIG. 1B shows a Western blot analysis to evaluate critical survival pathways known to contribute to drug resistance in plasma cell cancers were interrogated. The Western blot analysis demonstrated a shift in the protein profiles of BR models vs. WT cells, notably in Bcl-2 family proteins consistent with other reports of drug resistant multiple myeloma and other lymphomas. FIG. 1C is a sequencing readout suggesting that a mutation in the PSMB5 gene (G322A), which encodes an amino acid change (Ala to Thr) at position 108, may be involved in the mechanism of BR. Sequencing of the PSMB5 gene in all the BR models failed to demonstrate any mutation, suggesting a novel mechanism to be investigated.

FIGS. 2A-2C show that bortezomib resistant cells upregulate PSMB9 (1310 expression, which was localized within the 20S proteasome. FIG. 2A is a Western blot confirming a significant increase in production of β1i subunit protein in BR models (two representative cell lines) vs. their WT counterparts. An increase in β5 production also was noted in the BR state, suggesting an overall amplified proteasomal function. FIG. 2B is a picture of a co-localization assay showing that both PSMB5 (β5) and PSMB9 (β1i) are present within the proteasome. Protein extracts were immunoprecipitated with PSMA2 (proteasomal structural subunit) and probed by Western blot with anti-PSMB5 and anti-PSMB9 antibodies. The results show that both PSMB5 and PSMB9 are constituents of the proteasome in BR cells, a phenomenon that is not normally present in the wild type cells. FIG. 2C is a picture of a Western blot using tumor cells obtained from patients. CD138⁺ cells from bone marrow of MM patients resistant to bortezomib were isolated by magnetic separation, and protein extracts were analyzed for the expression of PSMB5 and PSMB9 by Western blotting. PSMB9 was highly expressed in all patients tested (n=6). Ten (10) mg of the protein were loaded for all samples, and equal protein loading was confirmed by GAPDH immunoblotting.

FIGS. 3A-3C are a series of graphs showing that bortezomib resistance is associated with increased proteasomal enzymatic activity. To investigate the functional significance of increased PSMB9 and PSMB5, 26S proteasome activity was measured by cleavage of a fluorogenic substrate (Suc-LLVY-AMC, β5/PSMB5 dependent), and was found to be significantly amplified in BR models (FIG. 3A). β1i/PSMB9 activity (as measured by Ac-PAL-AMC cleavage) also was significantly increased in BR cells (FIG. 3B). Notably, in the presence of bortezomib, chymotryptic β5 activity was decreased in both WT and BR cells alike (FIG. 3C), indicating target engagement by bortezomib, albeit without lethality (compare with FIG. 1A).

FIGS. 4A-4D show the characterization of BR WM models, established by subjecting WM cancer cell lines (n=3) chronic drug exposure. FIG. 4A is a graph plotting the results of an MTS assay in one representative model, the BCWM.1 cell line (IC₅₀=18 nM) and its corresponding BR derivative (BCWM.1/BR, IC₅₀>1000 nM), demonstrating complete resistance to bortezomib. FIG. 4B is a Western blot conducted to evaluate critical survival pathways known to contribute to drug resistance in NHLs, demonstrating modulation of several proteins in BCWM.1/BR cells relative to BCWM.1 cells—particularly pro and antiapoptotic proteins in the Bcl-2 family. FIG. 4C is a graph plotting relative fluorescence units in an assay to examine PSMB5 enzyme function by flow cytometry. Marked upregulation of PSMB5/β5 activity (Suc-LLVY-AMC cleavage) was observed in all BR cells as compared to their bortezomib-sensitive parental cells, an observation also noted at the protein level by Western blot analysis (representative blot shown below the graph). FIG. 4D is a graph plotting relative fluorescence units in an assay to evaluate PSMB9/β1i function. β1i catalytic activity (Ac-PAL-AMC cleavage) and protein levels were significantly upregulated in BR cells compared to their wild type parental cells or HCT-8 colon cancer cells (negative control).

FIG. 5 is a graph plotting sensitivity of bortezomib or carfilzomib-resistant tumor cells to drugs with a mechanism directed at targets upstream of the 20S proteasome. An inhibitor of the deubiquitinase (DUB) enzymes located in the 19S cap of the proteasome that are upstream of β5 and β1i, was used. While resistant to 20S proteasome inhibition, targeting upstream at the 19S proteasome-lid elicited comparable cytotoxicity in bortezomib/carfilzomib-resistant WM and MM cells.

FIGS. 6A-6D are a series of graphs indicating that increased PSMB9 mRNA and gene copy number are associated with poor clinical outcome in multiple myeloma (MM) patients. FIG. 6A is a graph plotting the clinical impact of PSMB9 RAN levels in 196 MM patients, showing that patients having higher PSMB9 expression did not demonstrate a clinically meaningful response to treatment (SD, stable disease; PD, progressive disease), and had no induction of remission. In contrast, patients having low PSMB9 expression were able to achieve remission (CR, complete remission). FIG. 6B is a graph plotting duration of response (DOR) to treatment (generally bortezomib-based therapy) for MM patients, separating those with a PSMB9 gene copy number gain from those with a PSMB9 gene copy number loss. FIGS. 6C and 6D are graphs plotting progression free survival (PFS) and overall survival (OS) for the MM patients, again separated by PSMB9 gene copy number gain or loss.

FIGS. 7A-7C show development of a novel mouse monoclonal antibody to β1i/PSMB9. FIG. 7A is a picture of an immunohistochemistry (IHC) blot confirming the antibody's specificity for β1i. FIG. 7B is a flow cytometry histogram and table, and FIG. 7C is a Western blot from OPM2/BR MM tumor cells transfected with either a scrambled shRNA (NTC) or PSMB9 shRNA plasmid (negative control). Notably, no PSMB9 band was noted in shRNA transfected cells, indicating specificity of the antibody for PSMB9.

FIG. 8 is a diagram indicating the potential of clinical impact of detecting PSMB9 in triaging proteasome based therapeutics.

FIGS. 9A and 9B show the structures of VLX1500 (FIG. 9A) and VLX1570 (FIG. 9B).

FIGS. 10A and 10B are a series of graphs plotting the effect of b-AP15 treatment on 20S proteasome, b5-subunit (chymotrypsin-like) catalytic activity in WT and BR WM cells. The effects of bortezomib (Bort, 10 nmol/l), carfilzomib (Carf, 10 nmol/l), and/or b-AP15 (10 nmol/l) on the proteasomal activity of WM cell lines was measured in vitro using fluorogenic substrates (chymotryptic activity, LLVY shown). b-AP15 did not alter chymotryptic activity or abrogate the ability of bortezomib or carfilzomib to disrupt the chymotrypsin-like activity in either WT BCWM.1, MWCL-1 or RPCI-WM1 cells (FIG. 10A), or in their BR subclones (FIG. 10B).

FIGS. 11A-11D are a series of Western blots and graphs showing that USP14 and UCHL5 are expressed in WM cells, and that their inhibition with b-AP15 results in accumulation of high molecular weight ubiquitinated protein and loss of cell viability. FIG. 11A is a Western blot analysis of protein expression for the USP14 and UCHL5 DUB enzymes in primary patient-derived WM cells (n=2, WM1; bortezomib-refractory and WM2; previously treated but bortezomib-naive) with and without b-AP15 (0.5 μmol/l), while FIG. 11B is a Western blot analysis of the same DUBs in WT and BR WM cell lines with and without b-AP15 treatment (0.5 μmol/l and 1 μmol/l). FIG. 11C is an immunoblot showing the effect of b-AP15 on the cellular content of ubiquitinated proteins. FIG. 11D is a pair of graphs from 72-h MTS assay conducted to assess WM cell viability after treatment with increasing concentrations of b-AP15 (0-1 μmol/l). MWCL-1 cells were more sensitive (IC₅₀ 7 nmol/l) than BCWM.1 (IC₅₀ 9 nmol/l) and RPCI-WM1 (IC₅₀ 16 nmol/l) (left panel). BR tumor cell viability was observed in a similar order. IC₅₀ of MWCL-1B/BR was lowest, at 3 nmol/1, followed by BCWM.1/BR (IC₅₀ 16 nmol/l) and finally RPCI-WM1/BR (IC₅₀ 57 nmol/l) (right panel).

FIGS. 12A-12D are a series of graphs and Western blots showing b-AP15 induction of tumor-specific apoptosis in WM cell lines and primary patient-derived WM cells. FIG. 12A is a graph plotting cell death as a percentage of control cell death for all available WM cell lines (n=6, WT and BR derivatives) after treatment with the indicated concentrations of b-AP15, followed by staining with annexin-V and propidium iodide and then flow cytometry to examine apoptosis. Annexin-V positivity (apoptosis) was significantly observed in b-AP15-treated WM cells by 12 hours in a dose-dependent manner (**P<0.005). Each experiment was conducted a minimum of three times with control cells (no drug treatment) showing a viability (annexin-V positive and propidium iodide-negative population) of >85%. Following treatment, the percentage of cells affected by b-AP15 was calculated by normalizing data from treated cells relative to the control (untreated) cells. FIG. 12B is a graph plotting apoptosis of malignant CD19+/CD138+WM cells from human patients (WM1 and WM2), and of peripheral blood mononuclear cells (PBMCs, n=2), stained with annexin-V and propidium iodide as in FIG. 12A. Robust apoptotic cell death was noted in patient-derived WM cells after a 12 hour exposure to b-AP15 (0.5 μmol/l). In contrast, minimal apoptosis (˜13%) was observed for b-AP15-treated PBMCs exposed to the deubiquitinase enzyme inhibitor for 48 hours. FIGS. 12C and 12D are pictures of immunoblots for PARP1 cleavage, confirming execution of apoptosis in both WM tumor cell lines (FIG. 12C) and primary WM tumor cells (FIG. 12D).

FIGS. 13A and 13B show that b-AP15 alters mitochondrial membrane permeability (MOMP) in WM cells. FIG. 13A is a graph plotting MOMP in WM cell lines and TMRM-negative cells, measured in relation to TMRM fluorescence and calculated to represent % MOMP (four representative cell lines shown). MOMP was significantly induced in b-AP15-treated WT and BR WM cells, and correlated with PARP1 cleavage as well as cleavage of executor caspase-3. FIG. 13B is a series of blots from experiments conducted to determine if b-AP15 mediated toxicity was caspase dependent. WM cell lines (two WT with respective BR subclones) were treated with the pancaspase inhibitor z.VAD.fmk±b-AP15. Pre-treatment of b-AP15 containing WM cells with z.VAD.fmk significantly reduced MOMP (**P<0.01), indicating that b-AP15 associated MOMP is partially caspase-dependent in WM cells.

FIGS. 14A and 14B are diagrams depicting genes altered in b-AP15 treated WM cells. BCWM.1 and RPCI-WM1 were treated with b-AP15 (50 nmol/l), and BR clones were treated with 100 nmol/1 of the DUB inhibitor for 24 hours, followed by collection of RNA for profiling using the NanoString nCounter assay. FIG. 14A is an intersect analysis in which treated (Tx) cell lines were first compared to their untreated counterparts and then against one another to delineate which genes were altered in the same orientation across all four cell lines tested. 36 genes were identified, and are listed in TABLE 2. FIG. 14B is a diagram of an IPA network analysis, depicting the relationship between the 36 genes and illustrating the interaction and relative expression of these genes. The darkness of the node color denotes the degree of differential gene expression as compared to baseline.

FIGS. 15A-15C are a graph and blots indicating that nuclear translocation of RELA (NF-κB p65) and its downstream target MYC are reduced by b-AP15 in WM cells. FIG. 15A is a graph plotting NFκB luciferase activity in HEK293 cells expressing MYD88L265P and treated with b-AP15 at the indicated doses. After 24 hours, luciferase activity was measured in cell extracts and normalized against Renilla. b-AP15 treatment resulted in significant reduction of NF-κB reporter activity (**P<0.004) in these cells. Results are from two independent experiments done in triplicate. Expression of cytoplasmic and nuclear RELA, as well as total and nuclear MYC, was determined by Western blot analysis in untreated and b-AP15 treated (6 hours) WM cells (BCWM.1 shown). As shown in FIG. 15B, RELA nuclear protein levels were markedly reduced after b-AP15 treatment, as were levels of its direct target, MYC (FIG. 15C).

FIGS. 16A and 16B are a series of Western blots showing that b-AP15 induces a shift in the protein profiles of WM cells. Experiments were focused mainly on markers of endoplasmic reticulum (ER) and cell stress-associated signaling. The blots in FIG. 16A show that the ER stress-associated protein HSPA1A was present in all cell lines and was further induced by b-AP15. Likewise, ERN1a, XBP1u (unspliced), and XBP1s (spliced) were significantly induced by b-AP15 across all models tested. The blots in FIG. 16B show that cell stress kinases also were modulated by an increase in p-MAPK3/MAPK1 (ERK1/2) in wild type cell lines after b-AP15. No change in BCL2 was noted, but a marginal increase in TP53 was observed in b-AP15 treated BCWM.1 and BCWM.1/BR WM cells.

FIGS. 17A-17D are a series of graphs plotting the effects of bortezomib (10 nM), carfilzomib (10 nM) and b-AP15 (10 nM) on caspase-like and trypsin-like proteasomal activities, as assessed in three WT and three BR WM cell lines in vitro using the fluorogenic substrates LLE-AMC (caspase-like activity) and LRR-AMC (trypsin-like activity). Reactions were incubated at 37° C. for 1 hour and the fluorescence was measured at 360/460 and expressed as relative fluorescence units (RFU), using BioTek synergy HT plate reader. Data from two representative cell lines (BCWM.1 and its BR subclone) are shown. Following b-AP15 treatment, no change in caspase-like activity was observed in either BCWM.1 cells (FIG. 17A) or BCWM.1/BR cells (FIG. 17B). Similarly, no change in trypsin-like activity was seen in b-AP15 treated BCWM.1 cells (FIG. 17C) or their BR subclones (FIG. 17D). In all co-treatment experiments (b-AP15±bortezomib or carfilzomib), b-AP15 treatment did not impact the effects of either PI on the enzymatic activities examined.

FIG. 18 is a series of representative heat density plots showing Annexin-V staining in BCWM.1 and BCWM.1/BR±b-AP15 treatment, indicating about 46% cell death in BCWM.1 cells (top panels) and about 42% cell death in BCWM.1/BR cells (bottom panels).

FIG. 19 is a pair of histograms showing MOMP in representative WM models±b-AP15 treatment (BCWM.1 and BCWM.1/BR cells; one WT with its respective BR subclone). Black histogram represents control. Lines represent a shift in MOMP. A greater increase in MOMP was observed in BCWM.1 cells (57%).

FIG. 20 is a graph indicating IPA generated canonical pathways that are enriched with genes from TABLE 2.

FIG. 21 is a graph plotting apoptotic cell death in bortezomib-sensitive or BR WM cells. Four WM cell lines (two WT, two BR) were treated with b-AP15 (0.5 μM), the p38 inhibitor SB580190 (10 μM), or the combination of b-AP15+SB580190 for 6 hours. Cells were stained with annexin-V, followed by flow cytometry for assessment of apoptosis. No significant change was observed in either WT or BR b-AP15-treated WM models with the addition of SB580190.

### DETAILED DESCRIPTION

This document provides methods and materials involved in identifying mammals having blood cancer (e.g., lymphoma, including WM, MCL, or DLBCL, or myeloma) resistant to treatment with a proteasome inhibitor such as bortezomib (e.g., VELCADE®). For example, this document provides methods and materials for using the expression level of PSMB9 nucleic acid to identify a mammal as having a blood cancer (e.g., myeloma or lymphoma, such as WM, MCL, or DLBCL) resistant to treatment with a proteasome inhibitor such as bortezomib (e.g., VELCADE®). Any appropriate mammal can be assessed for resistance to treatment with a proteasome inhibitor such as bortezomib as described herein. For example, dogs, cats, horses, cows, pigs, sheep, goats, monkeys, and humans can be assessed for resistance to treatment with a proteasome inhibitor such as bortezomib.

As described herein, the presence of an elevated level of expression of PSMB9 nucleic acid within blood cancer cells (e.g., myeloma or lymphoma cells, including WM, MCL, or DLBCL cells) from a mammal can indicate that that mammal (e.g., a human) has a blood cancer resistant to a proteasome inhibitor that targets the β5 subunit of a proteasome such as bortezomib, and may exhibit shorter duration of response to treatment with bortezomib-based therapy, lower progression free survival, and/or lower overall survival. The term “elevated level” as used herein with reference to the expression level of PSMB9 nucleic acid refers to an increased level of PSMB9 mRNA or β1i polypeptides as compared to the level of PSMB9 mRNA or β1i polypeptides present within normal, non-cancerous control cells (e.g., plasma cells or lymphoid cells). In some cases, an elevated level of PSMB9 nucleic acid expression can be 5, 10, 20, 30, 40, 50, or 75 percent more than that observed in normal, non-cancerous control cells (e.g., plasma cells or lymphoid cells). Any appropriate blood cell sample can be used as described herein to identify mammals having blood cancer (e.g., myeloma or lymphoma, such as WM, MCL, or DLBCL) resistant to treatment with a proteasome inhibitor such as bortezomib (e.g., VELCADE®). For example, blood samples, bone marrow samples, and tumor cell samples can be used to determine whether or not a mammal has an elevated level of PSMB9 nucleic acid expression.

Once obtained, a sample (e.g., a blood sample) can be processed prior to measuring a level of expression. For example, a blood cell sample can be processed to extract RNA from the sample. Once obtained, the RNA can be evaluated to determine the level of PSMB9 mRNA. In some cases, nucleic acids present within a sample can be amplified (e.g., linearly amplified) prior to determining the level of expression (e.g., using array technology or RNA-sequencing).

Any appropriate method can be used to determine the level of expression of PSMB9 mRNA within a sample. For example, quantitative real time PCR, in situ hybridization, microarray technology, or RNA-sequencing can be used to determine whether or not a particular sample contains an elevated level of PSMB9 mRNA expression or lacks an elevated level of PSMB9 mRNA expression. In some cases, the level of PSMB9 nucleic acid expression can be determined using polypeptide detection methods such as immunochemistry or flow cytometry techniques. For example, antibodies specific for β1i polypeptides can be used to determine the level of β1i in a sample. In some cases, polypeptide-based techniques such as ELISAs and immunocytochemistry techniques can be used to determine whether or not a particular sample contains an elevated level of β1i polypeptides or lacks an elevated level of β1i polypeptide.

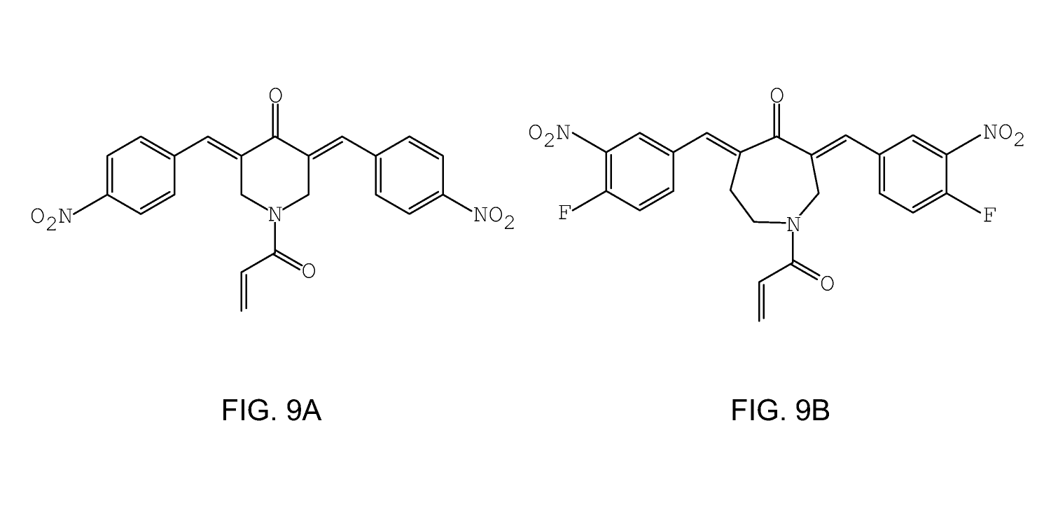

Once the level of PSMB9 expression in a sample is determined, the level can be compared to a reference level (e.g., the expression level observed in control samples) and used to classify the mammal as being susceptible or resistant to treatment with a proteasome inhibitor such as bortezomib. For example, the presence of an elevated level of PSMB9 nucleic acid expression can indicate that the mammal is resistant to treatment with a proteasome inhibitor such as bortezomib, while the absence of an elevated level of PSMB9 nucleic acid expression can indicate that the mammal is susceptible to treatment with a proteasome inhibitor such as bortezomib. Mammals identified as being resistant to treatment with a proteasome inhibitor such as bortezomib can be treated with carfilzomib (e.g., KYPROLIS®) or other drugs that can bypass β1i and β5 (such as VLX1570; FIG. 9B). Mammals identified as being susceptible to treatment with a proteasome inhibitor such as bortezomib can be treated with bortezomib or can continue to be treated with bortezomib.

This document also provides methods and materials for treating blood cancer (e.g., myelomas or lymphomas, such as WM, MCL, and DLBCL). For example, a mammal having a blood cancer (e.g., myeloma or lymphoma, including WM, MCL, or DLBCL) and being treated with bortezomib can be assessed for cancer cells expressing an elevated level of PSMB9 nucleic acid. This assessment can be performed once during or after the bortezomib treatment or can be performed periodically during and/or after bortezomib treatment. For example, this assessment can be performed one or more (e.g., two, three, four, five, or more) times and as frequently as every month during bortezomib treatment. Once the mammal is determined to have cancer cells with an elevated level of PSMB9 nucleic acid expression, then the mammal can be administered carfilzomib (e.g., KYPROLIS®), or other drugs that can bypass β1i and β5 (such as VLX1570), and treatment with bortezomib can be stopped. In some cases, the mammal identified as having cancer cells with an elevated level of PSMB9 nucleic acid expression can remain on a treatment with bortezomib while also being treated with carfilzomib (e.g., KYPROLIS®) other drugs that can bypass β1i and β5 (such as VLX1570).

In some embodiments, the compound useful in the methods provided herein can be an analog of VLX1500 (FIG. 9A), such that it is structurally similar to VLX1500 but differs slightly in composition (e.g., by replacement of one or several atoms or functional groups with an atom of a different element or a different functional group, or by adding or removing one or more functional groups). VLX1570 (FIG. 9B) is an exemplary analog of VLX1500.

The invention will be further described in the following examples, which do not limit the scope of the invention described in the claims.

### EXAMPLES

### Example 1—Development and Characterization of Bortezomib-Resistant Tumor Cells

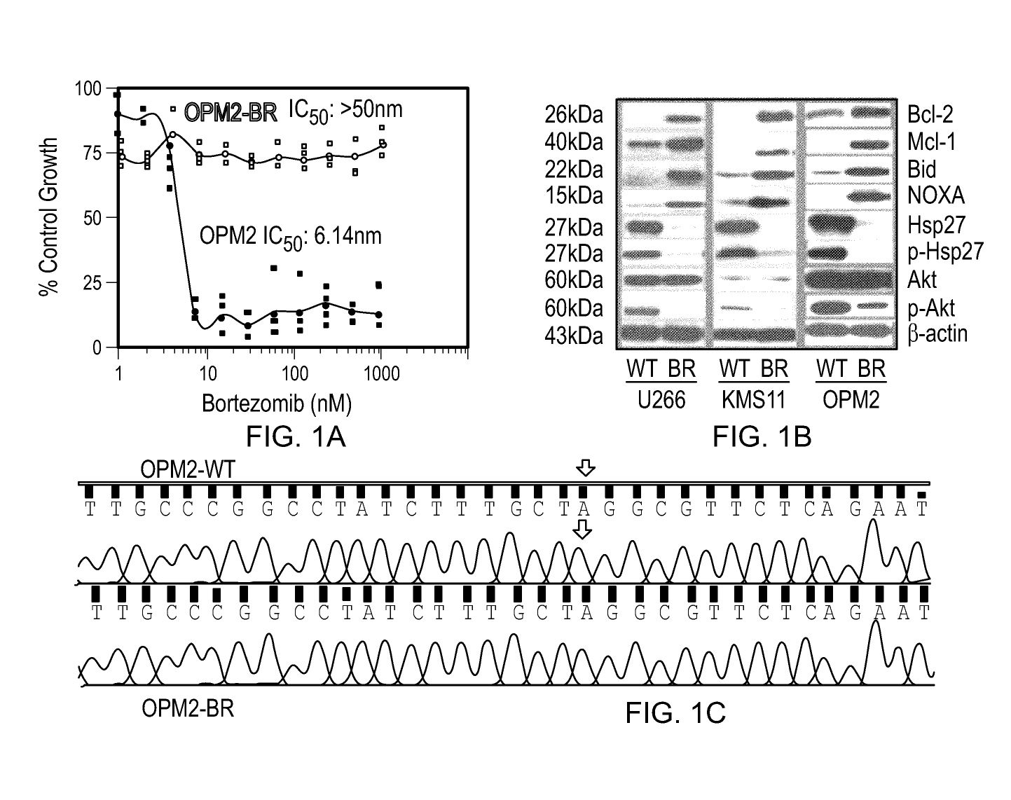

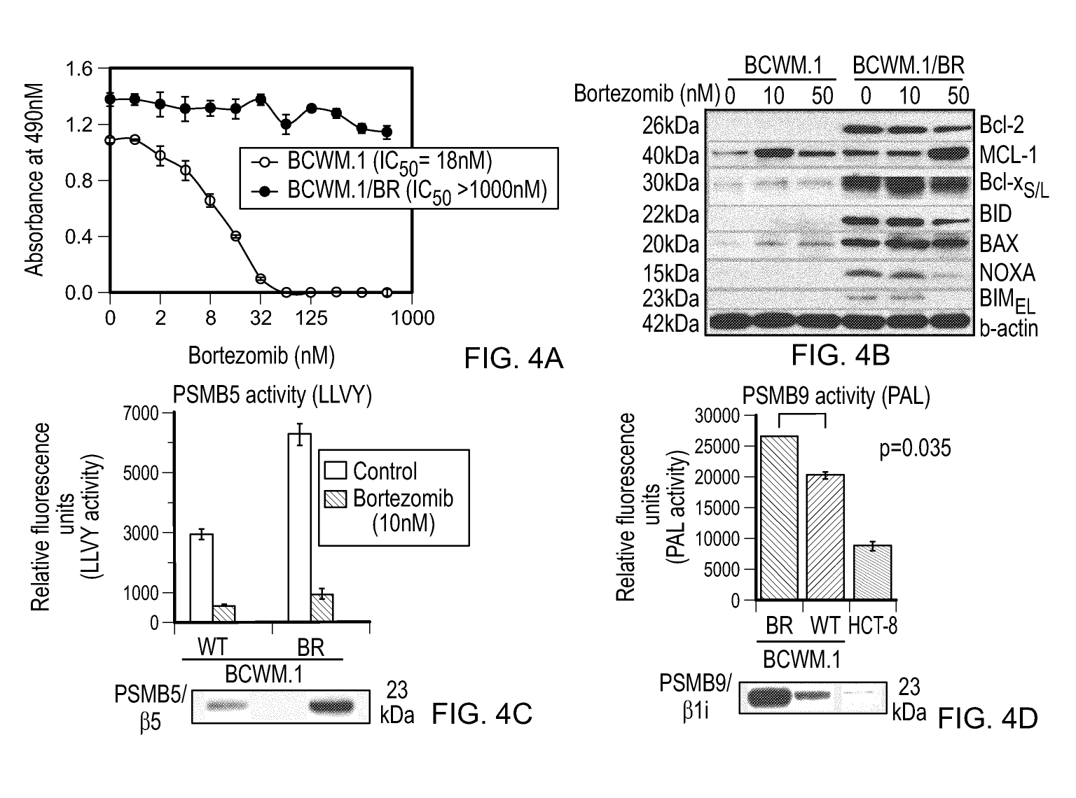

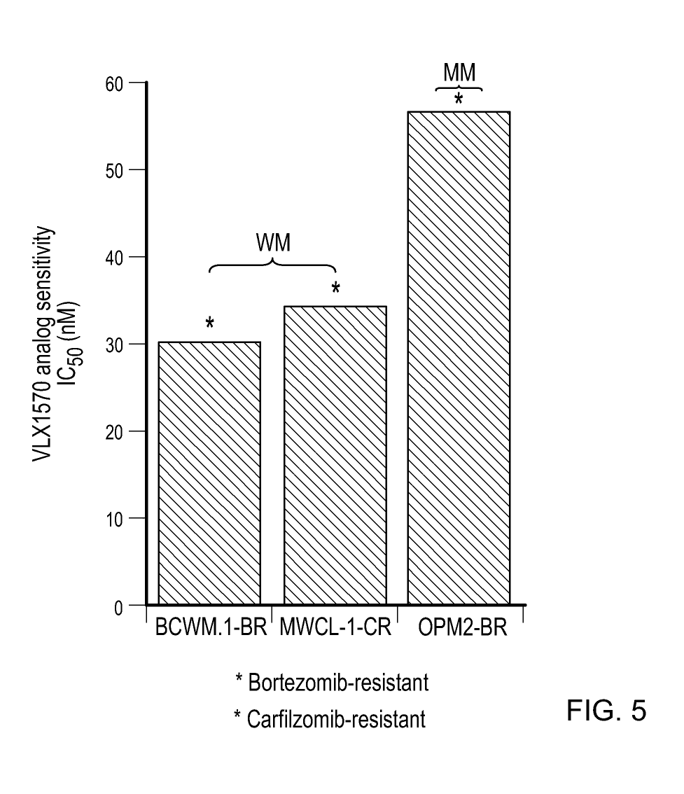

To investigate the mechanism of induction of bortezomib resistance, preclinical models of acquired bortezomib resistance (BR) were developed using human MM and WM cell lines. BR cell lines demonstrated >100-fold increase in growth insensitivity to bortezomib treatment as compared to their respective WT counterparts (FIG. 1A, representative cell line). Pathways critical to malignant B-cell survival were than surveyed, and a shift in expression of pro-survival proteins belonging to the Bcl-2 and Akt family was noted (FIG. 1B). These proteins were previously reported to contribute to bortezomib-insensitivity (Chitta et al., ASH Annual Meeting Abstracts, 114(22):4919 (2009); Paulus et al., Br. J. Haematol., 164(3):352-365 (2014); and Buda et al., Annals of Hematology, 89(11):1133-1140 (2010), however, inhibition of these pathways did not completely explain mechanism of bortezomib resistance, warranting continued investigation. Mutations in the PSMB5 gene also were evaluated (Siegel et al., CA Cancer J. Clin., 62(1):10-29 (2012); and Ri et al., Leukemia, 24(8):1506-1512 (2010)). Intriguingly, sequencing of the specific region (G322) in the BR models did not demonstrate any mutation (FIG. 1C, representative model). Thus, it was hypothesized that in the absence of this (or any) mutation the catalytic activity of β5 could still be inhibited by bortezomib. Indeed, functional analysis confirmed that bortezomib inhibited the chymotryptic activity even in the BR state and comparable to that in the WT cells, but this was no longer lethal to the cell. These results suggest that an alternative mechanism(s) of resistance to bortezomib may be independent of β5 function. Similar observations were observed in bortezomib-resistant WM cells (FIGS. 4A and 4B). PSMB5 G322A mutation analysis carried out by whole exome sequencing (WES) and Sanger sequencing of the PSMB5 gene demonstrated no mutations in all WM BR models.

### Example 2—Proteasome Subunits β1i and β5 are Overexpressed in Bortezomib-Resistant MM and WM Cells

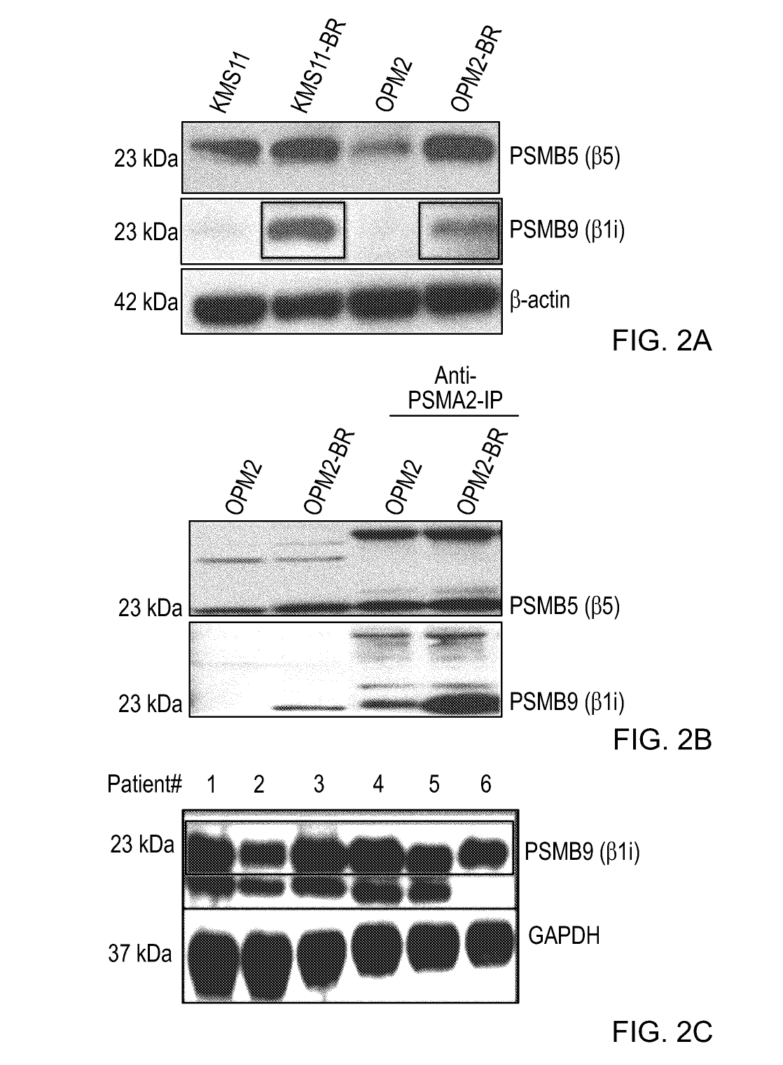

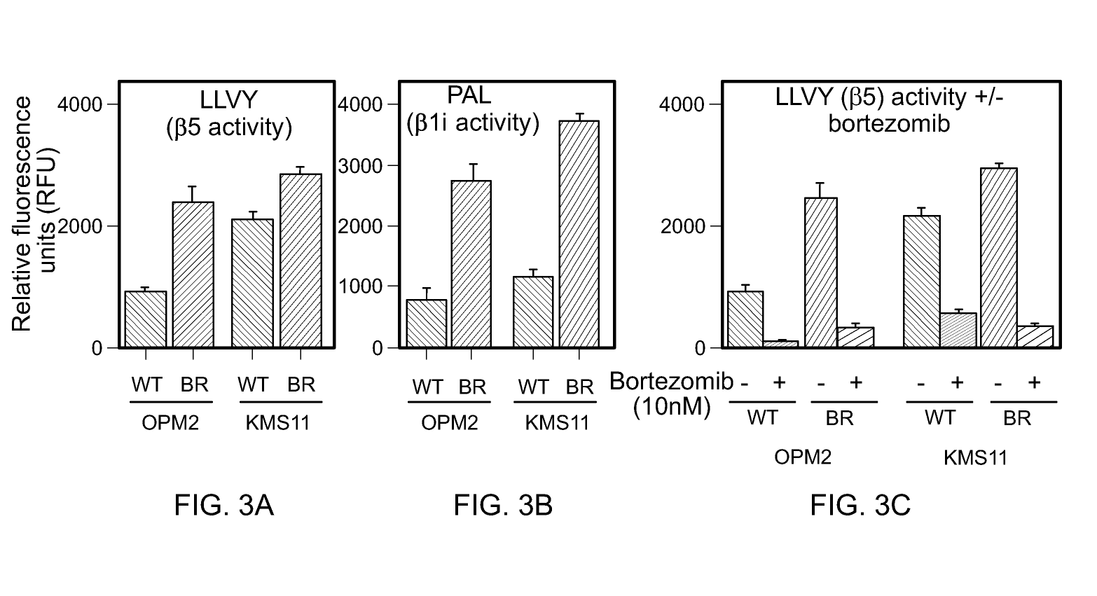

Global gene expression analysis of the BR models uncovered upregulation of the PSMB9 gene. Immunoblot assay confirmed significant increase in its protein product in BR vs. WT cell lines (FIGS. 3A and 4D). Further Western blotting confirming a significant increase in production of β1i subunit protein in BR models vs. their WT counterparts (FIG. 2A). An increase in β5 production also was noted in the BR state, suggesting an overall amplified proteasomal function.

Next, experiments were performed to determine whether increased β1i and β5 subunits were present within the same proteasome. Protein extracts were immunoprecipitated with PSMA2 (proteasomal structural subunit) and probed by Western blot with anti-PSMB5 and anti-PSMB9 antibodies. In the BR cells, both subunits co-localized to PSMA2 (FIG. 2B), demonstrating presence of the hybrid proteasome phenotype with dual β1i (PSMB9) and β5 (PSMB5) catalytic activities. Existence of such hybrid proteasome was previously reported (Guillaume et al., PNAS, 107(43):18599-18604, 2010; and Guillaume et al., J. Immunol., 189(7):3538-3547, 2012), but not in context with, or as a potential mechanism of, acquired resistance to bortezomib.

Patient tumor cells obtained from patients were then examined. CD138+ cells from bone marrow of MM patients who were resistant to bortezomib were isolated by magnetic separation, and protein extracts were analyzed for the expression of PSMB5 and PSMB9 by Western blotting. PSMB9 was highly expressed in all patients tested (n=6) (FIG. 2C). Ten (10) mg of the protein were loaded for all samples, and equal protein loading was confirmed by GAPDH immunoblotting.

To validate whether the observation in BR models was clinically relevant, PSMB9/β1i expression was examined in CD138⁺ malignant plasma cells from MM patients, who were noted to have acquired resistance to bortezomib after treatment. Significantly increased PSMB9/β1i product levels were observed in the BR patients' tumor cells (FIG. 3C), validating that PSMB9/β1i up-regulation was associated with BR. These results demonstrate that one of the consequences of resistance to bortezomib is acquisition of the β1i proteasomal catalytic subunit within the 26S proteasome.

### Example 3—Bortezomib Resistance is Associated with Increased Proteasomal Enzymatic Activity

Studies were performed to investigate whether an increase in β1i and β5 proteins corresponded with increased protease activity. WT and BR cancer cells were incubated with β1i specific (Ac-PAL-AMC substrate) and β5 specific (Suc-LLVY-AMC substrate) fluorogenic peptides (condition details in FIG. 3 description above). β5 chymotryptic activity (primary target of bortezomib) was assessed first and found in BR cells. LLVY cleavage was markedly increased relative to WT cells (FIG. 4C). β1i enzymatic activity was assessed next. PAL cleavage was notably more evident in BR myeloma and WM derivatives than WT parental cells (FIGS. 3B and 4D). Interestingly, in the presence of bortezomib, β5 activity was nearly abrogated in BR cells in a similar manner to the WT counterparts (FIG. 3C). This indicates that (1) proteasomal chymotryptic activity is intact and in fact enhanced in a BR state and (2) bortezomib continues to engage its target in BR cells and lowers the chymotryptic action, albeit without causing a lethal effect, in contrast to WT (compare FIGS. 3C and 1A). Taken in concert, these results demonstrate the continued functional importance of both PSMB5 (β5) and PSMB9 (β1i) in a BR state and the cells' ability to amplify the proteasome functionality through development of a mutant parallel system that incorporates a new subunit in the catalytic cylinder.

### Example 4—Treating Blood Cancers

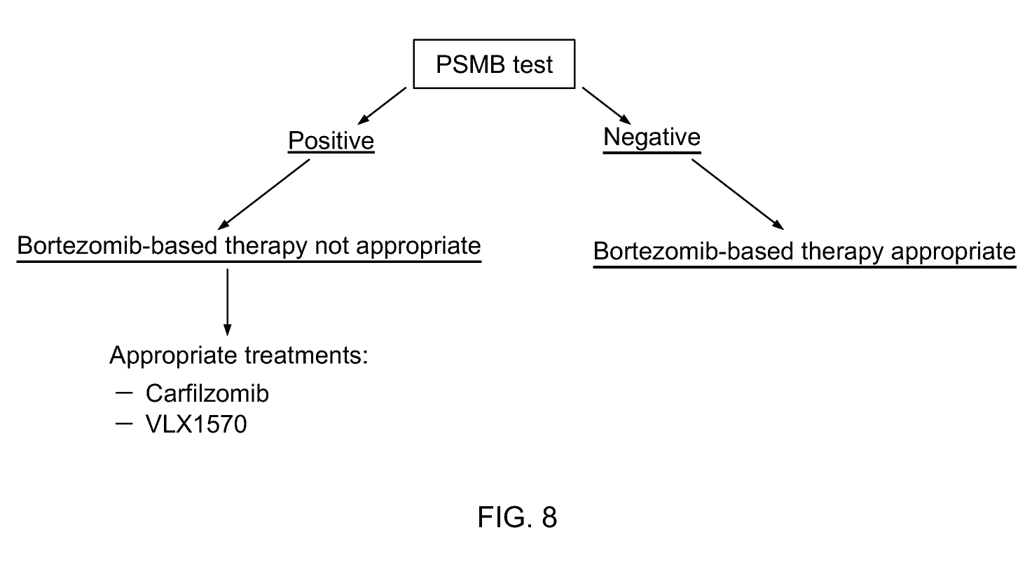

A cancer patient is identified as having a blood cancer (e.g., myeloma or lymphoma, such as WM, MCL, DLBCL). Once identified, the patient is treated with bortezomib (e.g., VELCADE®). At various time point during and/or after the treatment course with bortezomib, samples are collected from the patient and assessed for an increased expression of PSMB9 nucleic acid. An increased level of expression PSMB9 nucleic acid can be determined by assessing mRNA levels or polypeptide levels (e.g., β1i polypeptide levels). When an increased level of PSMB9 nucleic acid expression is detected (PSMB9 test positive), the patient is no longer treated with bortezomib and instead is treated with carfilzomib (e.g., KYPROLIS®) or XLV1570 (FIG. 5). When PSMB9 nucleic acid expression is low (PSMB9 test negative), the patient may be treated with bortezomib-based therapy (FIG. 8). This algorithm is set out in the schematic shown in FIG. 8.

The sensitivity of bortezomib or carfilzomib-resistant tumor cells to drugs whose mechanism is directed at targets upstream of the 20S proteasome (and thus bypassing the catalytic sites (β5 and 1310 in the 20S proteasome) is indicated by FIG. 5. DUB functioning is as critical as β5 to proteasome function. A VLX1570-sensitivity screen (Celltiter Glo viability assay) was performed in bortezomib or carfilzomib-resistant MM and WM cell lines. Although resistant to 20S proteasome inhibition, targeting upstream at the 19S proteasome-lid elicited comparable cytotoxicity in bortezomib/carfilzomib-resistant tumor cells. This demonstrated that identification of β1i/PSMB9 engagement can be clinically important in order to triage patients away from β5 targeting agents that will have low or no likelihood of meaningful clinical impact with continued use of the agents (bortezomib), and thus can be triaged to drugs with alternative mechanism(s).

### Example 5—Increased PSMB9 Affects Clinical Outcome in MM Patients

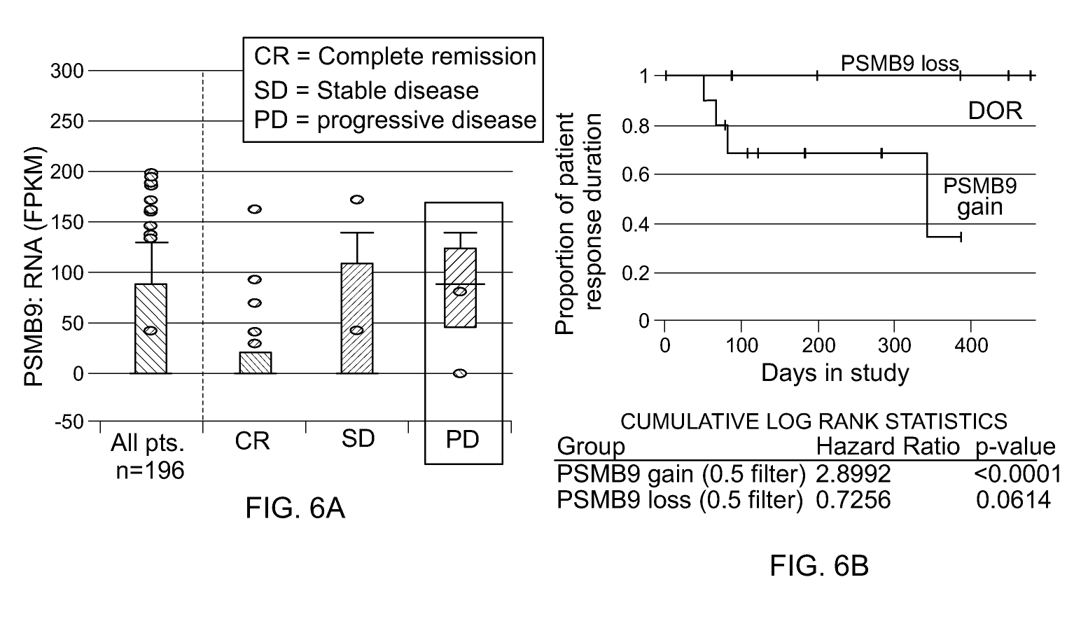

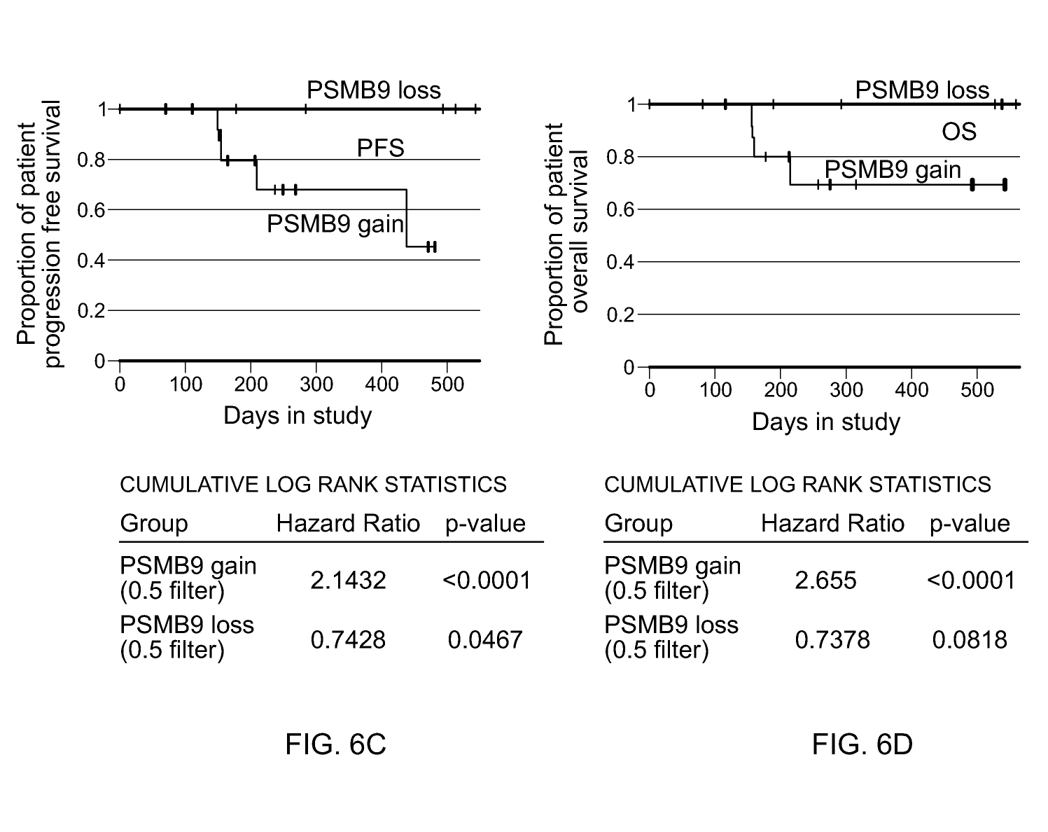

To evaluate the clinical implication of detecting β1i/PSMB9, a larger analysis was conducted using patient derived data. The Multiple Myeloma Research Foundation (MMRF) COMMPASS database, which contains genomic and clinical characteristics for over 196 MM patients, was queried to determine clinical impact of PSMB9. Interrogation of this robust data demonstrated that patients with higher PSMB9 expression failed to optimally benefit from treatment given, with no clinically meaningful response as defined by SD (stable disease) and PD (progressive disease) without induction of remission (FIG. 6A). In contrast, patients with low PSMB9 expression were able to achieve remission (CR; complete remission). Further analysis demonstrated that MM patients with a PSMB9 gene copy number gain had a significantly lower duration of response (DOR) to treatment, which for the majority of patients consisted of bortezomib-based therapy (FIG. 6B). PSMB9 copy number gain also was associated with lower progression free survival (PFS; FIG. 6C) and lower overall survival (OS; FIG. 6D).

### Example 6—Antibody Against β1i/PSMB9

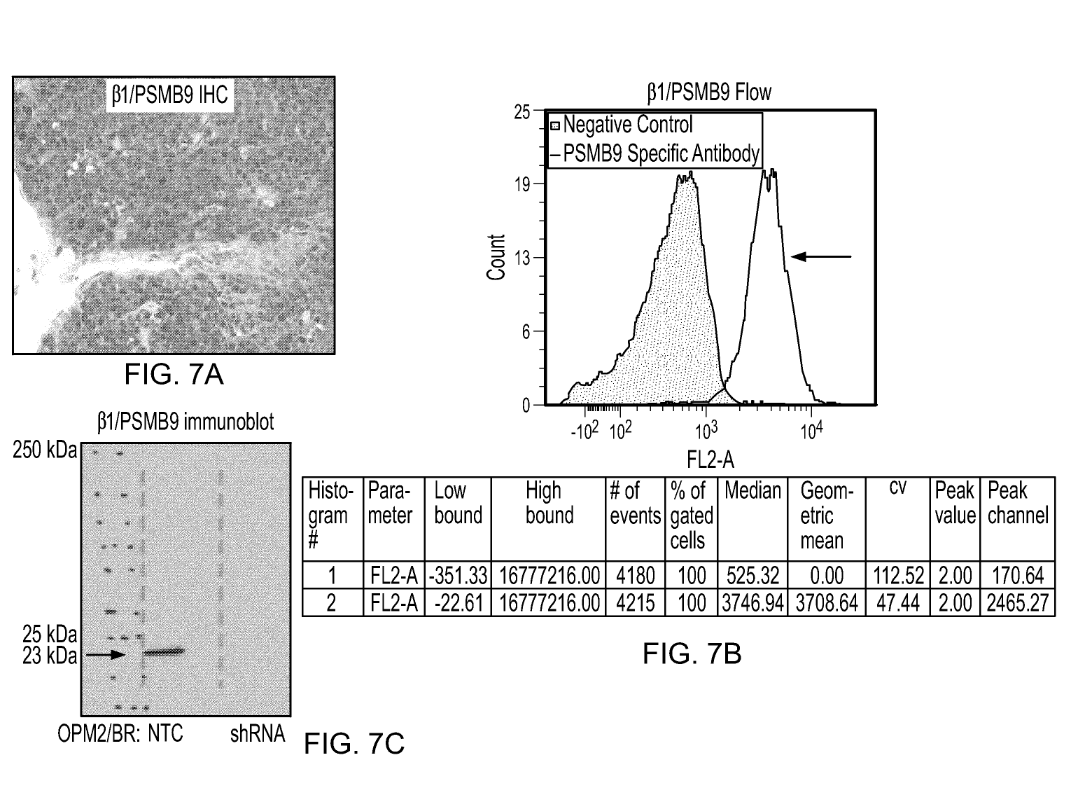

To effectively and dependably detect β1i expression in tumor cells, a mouse monoclonal antibody to the β1i/PSMB9 protein raised and developed. Confirmation of its specificity for β1i was obtained by immunohistochemistry (IHC; FIG. 7A), flow cytometry (FIG. 7B), and Western blot analysis (band at 23 kD) in OPM2/BR MM tumor cells transfected with either scrambled shRNA (NTC) or PSMB9 shRNA plasmid (negative control) (FIG. 7C). Notably, no PSMB9 band was noted in shRNA transfected cells, indicating the specificity of the antibody for PSMB9.

### Example 7—Targeted Inhibition of USP14 and UCHL5 in WM Tumor Cells

### Materials and Methods

Cell Lines, Cell Culture and Reagents:

Waldenstroms macroglobulinemia cell lines (BCWM.1, MWCL-1 and RPCI-WM1) and their corresponding bortezomib resistant (BR) clones [BCWM.1/BR, MWCL-1/BR and RPCI-WM1/BR, resistance of representative model shown in FIG. 4A and 50% inhibitory concentration (IC₅₀) of others in TABLE 1 were used in experiments as described elsewhere (Chitta et al., Blood (ASH Annual Meeting Abstracts), 114:2861, 2009; Paulus et al., supra). CD19+/CD138+ sorted tumor cells obtained from consenting WM patients were acquired from the Predolin Biobank (Mayo Clinic, Rochester, Minn.). Heparinized peripheral blood was obtained from healthy human donors. Peripheral blood mononuclear cells (PBMCs) from healthy human donors were isolated as described elsewhere (Chitta et al., Leukemia Lymphoma 55:652-661, 2014). VLX1500 (also referred to herein as b-AP15) was provided by Vivolux AB, (Uppsala, Sweden). Bortezomib and carfilzomib were purchased from Sellekhem (Houston, Tex., USA). RPMI medium, penicillin, streptomycin, tetramethylrhodamine, methyl ester (TMRM) and fetal bovine serum (FBS) were purchased from Life Technologies (Carlsbad, Calif., USA). All antibodies were purchased from Santa Cruz biotechnology (Dallas, Tex., USA) or Cell Signaling Technology (Danvers, Mass., USA). Annexin-V and propidium iodide apoptosis staining kit was purchased from BD Biosciences (San Jose, Calif., USA).

Proteasomal Activity Assay:

Cells were lysed at 4×10⁶ cells/ml in proteasomal activity assay buffer [assay buffer; 25 mmol/1 HEPES buffer, pH7.5 containing 0.5 mmol/1 EDTA, 0.05% Nonidet P-40, 0.01% sodium dodecyl sulfate (SDS)] and immediately used in the assay. Enzyme reactions were performed in 96-well plates with 100 μl of final volume containing 5 mmol/1 fluorogenic peptide substrates. The substrates used were LLVY-AMC for chymotrypsin like activity, LLE-AMC for caspase-like activity and LRR-AMC for trypsin-like activity. Reactions were incubated at 37° C. for 1 hour and fluorescence was measured at 360/460 using a BioTek synergy HT plate reader (BioTek, Winooski, Vt.).

Viability Assay:

Twenty thousand cells/200 μl in quadruplicates were incubated with serially diluted b-AP15 (1-1000 nmol/l) in 96-well plates at 37° C. for 72 hours. MTS (3-(4,5-dimethylthiazol-2-yl)-5-(3-carboxymethoxyphenyl)-2-(4-sulfophenyl)-2H-tetrazolium) reagent (Molecular Probes, Eugene, Oreg.) was added at 40 μl/well and the plates were further incubated at 37° C. for 3 hours and the developed color was read at 490 nm using a BioTek synergy HT plate reader against blanks with no cells.

Apoptosis Assay:

Apoptosis was measured using the Annexin V binding assay kit from BD Biosciences according to the manufacturer's instructions. Briefly, at the end of the treatment, cells were washed with PBS and 1×10⁶ cells were re-suspended in 100 μl of binding buffer. Fluorescein isothiocycanate (FITC)-labelled Annexin V (5 μl) and propidium iodide (10μ were added to each sample and incubated in the dark for 15 minutes at room temperature. The cells were subsequently analyzed by flow cytometry using BD Accuri, the C6 flow cytometer and its software. Data from 10,000 events per sample were collected and analyzed.

Determination of Mitochondrial Outer Membrane Permeability:

Cells were treated with different agents for 48 hours and assessed for MOMP using tetramethylrhodamine methyl ester [TMRM] (Life Technologies). TMRM was directly added to the cell cultures at 100 nmol/1 concentrations and incubated at 37° C. in the dark for 15 minutes. At the end of the incubation, cells were washed twice with cold PBS containing 2% FBS and analyzed. The cells were washed for fluorescence (FL2) and analyzed by BD Accuri, the C6 flow cytometer and its software. Data from at least 20,000 events per sample were collected and analyzed. TMRM-negative (%) cells were calculated to determine % MOMP.

Immunoblot Analysis:

Total protein extracts were made using radioimmunoprecipitation assay lysis buffer (50 mmol/1 Tris containing 150 mmol/1 NaCl, 0.1% SDS, 1% TritonX-100, 1% sodium deoxycholate, pH 7.2) with 0.2% protease and phosphatase inhibitor cocktail (Sigma, St. Louis, Mo.) on ice for 40 minutes, vortexing for 5 seconds every 10 minutes. Following centrifugation at 18,400 g for 20 minutes, the supernatant was collected and used for Western blot analyses. Protein content in the extracts was measured by bicinchoninic acid protein assay reagent. Aliquots of 20 μg of total protein were boiled in Laemmli sample buffer, loaded onto 10% SDS-PAGE gels, and transferred onto a nitrocellulose membrane. Membranes were blocked for 1 hour in TBS/Tween 20 [TTBS] containing 1% nonfat dried milk and 1% BSA. Incubation with primary antibodies was done overnight at 4° C., followed by washing 3× with TTBS and incubation for 1 hour with HRP-conjugated secondary antibody. The blots were developed using chemiluminescence (Thermo Scientific, Rockford, Ill.).

NE-κB Reporter Assay:

HEK293 cells expressing MYD88 L265P were generated as described elsewhere (Ansell et al., Blood Cancer J, 4:e183, 2014). Cells were transiently transfected with 0.5 ng Renilla and 0.25 μg of a pNFκB-luciferase reporter plasmid/1.0×10⁶ cells. b-AP15 was added to each well at the indicated doses; after 24 hours, luciferase activity was measured in cell extracts and normalized against Renilla with the Dual Luciferase Kit (Promega, Madison, Wis.).

Computational Docking:

Initial docking for bAP-15 was performed using Glide (v. 5.6) within the Schrödinger software suite (Schrödinger, LLC) (Mohamadi et al., J Comput Chem 11:440-467, 1990). The starting conformation of ligands was obtained by the method of Polak-Ribière conjugate gradient (PRCG) energy minimization with the Optimized Potentials for Liquid Simulations (OPLS) 2005 force field (Jorgensen and Tiradorives, J Am Chem Soc 110:1657-1666, 1988) for 5000 steps, or until the energy difference between subsequent structures was less than 0.001 kJ/mol-Å (ref. 1). Docking methodology has been described elsewhere (Caulfield and Devkota, Proteins 80:2489-500, 2012; Loving et al., J Comput Aided Mol Des 23:541-54, 2009; and Vivoli et al., Mol Pharmacol 81:440-454, 2012), as has the scoring function (Friesner et al., J Med Chem 49:6177-96, 2006). Briefly, in order to generate the grids for docking, molecular refracting molecules were removed from the USP14 or UCHL5 crystal structure (PDB Codes: 2AYO(Hu et al., Embo J 24:3747-56, 2005) and 3IHR (Burgie et al., Proteins 80(2):649-654, 2012), respectively). Schrödinger's SiteFinder module focused the grid on the active site region surrounding residues Cys114 and Cys88 for USP14 and UCHL5, respectively. Using this grid, initial placement for bAP-15 was docked using the Glide algorithm within the Schrodinger suite as a virtual screening workflow (VSW). The docking proceeded from lower precision through SP docking and Glide extra precision (XP) (Glide, v. 5.6, Schrödinger, LLC) (Salam et al., J Chem Inf Model 49:2356-68, 2009; and Caulfield and Medina-Franco, J Struct Biol 176:185-191, 2011). The top seeded poses were ranked for best scoring pose and unfavorable scoring poses were discarded. Each conformer was allowed multiple orientations in the site. Site hydroxyls, such as in serine and threonine residues, were allowed to move with rotational freedom. Docking scores were not retained as useful, since covalent bonding was the outcome. Thus, a covalent docking method was utilized within Schrödinger suite to allow the aldehyde of the reversible/irreversible inhibitor to form linkage to the thiol at the —SH group via a 1,4-Michael's addition reaction. Hydrophobic patches were utilized within the VSW as an enhancement. Top favorable scores from initial dockings yielded ˜10 poses with the top pose selected. XP descriptors were used to obtain atomic energy terms like hydrogen bond interaction, electrostatic interaction, hydrophobic enclosure and pi-pi stacking interaction that result during the docking run (Salam et al., supra; and Caulfield and Medina-Franco, supra). Molecular modeling for importing and refining the X-ray structure and generation of bAP-15 small molecule structures, as well as rendering of figure images were completed with Maestro, the built-in graphical user interface of the Schrödinger chemistry package (v. 5.6) (Schrödinger, LLC).

Molecular Dynamics Simulation (MDS):

MDS was completed on each model for conformational sampling, using methods described elsewhere (Caulfield and Devkota, supra; Caulfield et al., J Biophys Article ID 219515, 2011; Jorgensen et al., J Chem Phys 79:926-935, 1983; Reblova et al., Biophys J 93:3932-3949, 2007; and Reblova et al., Biopolymers 82:504-520, 2006). Following equilibration, the system was allowed to run MD calculations for approximately 50 nanoseconds in length. The primary purpose of MD for this study was conformational variability that may occur at the USP14 site where bAP-15 covalently binds. Charmm27 and OPLS2005 force fields were examined with the current release of NAMD2. The protein with hydrogens consists of 6,200 atoms. In all cases, counter-ions were used to neutralize, and a solvent was created with 150 mM Na+Cl− to recreate physiological strength. TIP3P water molecules were added around the protein at a depth of 12-15 Å from the edge of the molecule depending upon the side (Jorgenson et al., supra). The protocol has been described elsewhere (Caulfield et al., supra). Solvated protein simulations consist of a box with between 0.51×10⁵ atoms including proteins, counter-ions, solvent ions and solvent waters. Simulations were carried out using the particle mesh Ewald technique (PME) with repeating boundary conditions with a 9 Å nonbonded cut-off, using SHAKE with a 2-fs timestep. Pre-equilibration was started with 100,000 steps of minimization followed by 10000 ps of heating under MD, with the atomic positions of protein fixed. Then, two cycles of minimization (100000 steps each) and heating (2000 ps) were carried out with restraints of 10 and 5 kcal/(mol·Å2) applied to all protein atoms. Next, 50000-steps of minimization were performed with solute restraints reduced by 1 kcal/(mol·Å2). 1000 ps of unrestrained MD were carried out, and the system was slowly heated from 1 to 310 K. The production MD runs were carried out with constant pressure boundary conditions (relaxation time of 1.0 ps). A constant temperature of 300 K was maintained using the Berendsen weak-coupling algorithm with a time constant of 1.0 ps. SHAKE constraints were applied to all hydrogens to eliminate X-H vibrations, which yielded a longer simulation time step (2 fs). The methods for equilibration and production run protocols are consistent with those described elsewhere (Caulfield and Devkota, supra; Caulfield et al., supra; Jorgensen et al., supra; Reblova et al. 2007, supra; and Reblova et al. 2006, supra). Equilibration was determined from a flattening of RMSD over time after an interval of >20 ns. Translational and rotational center-of-mass motions (CoM) were initially removed. Periodically, simulations were interrupted to have the CoM removed again by a subtraction of velocities to account for unwanted translational-rotational motion. Following the simulation, the individual frames were superposed back to the origin, to remove rotation and translation effects.

RNA Preparation:

Total RNA from four WM cell lines (BCWM.1, BCWM.1/BR, RPCI-WM1 and RPCI-WM/BR) were prepared using Exiqon miRCURY RNA isolation kit (Exiqon, Woburn, Mass. USA) following the manufacturer's instructions. RNA samples were quantitated using a ND-1000 spectrophotometer (NanoDrop) and evaluated for degradation using a 2100 Bioanalyzer (Agilent Technologies, Santa Clara, Calif., USA).

Gene Expression Profiling Using the Nanostring nCounter Assay:

The NanoString nCounter (NanoString, Seattle, Wash.) assay was used for mRNA quantification and expression in WM cells that were treated with b-AP15, as described elsewhere (Geiss et al., Nat Biotechnol 26:317-25, 2008; and Paulus et al., Brit J Haematol 164:352-65, 2014). Briefly, a library is constructed with two sequence-specific probes for the gene of interest. Unique pairs of 3′ reporter and 5′ capture probes are developed to distinguish transcripts for the gene of interest using a color-based barcoding system. The capture probe contains a base pair sequence that is complementary to the target mRNA and linked to a biotin affinity tag, which binds the mRNA of interest. The reporter probe also is also designed to be complementary to the target mRNA, contains a base sequence that is linked to an RNA-based color-coded molecular tag that provides a signal for detection. Using this method, a distinct color code is digitally generated for each gene of interest. In our experiments, all probes were mixed together with total RNA (100 ng from each sample) in a single hybridization reaction for 12 hours at 65° C. in solution. Using the nCounter Prep Station, each probe-mRNA complex was captured post-hybridization onto a streptavidin coated surface, aligned and imaged. Each sample was scanned for 600 FOV on the nCounter Digital Analyzer. As each target mRNA is designated by its unique color code, the level of expression was quantified by counting the number of codes for each molecule. Subsequent normalization of the raw data to internal controls provided by the manufacturer was performed using the nSolver Analysis software v1.1. Data was extracted using the nCounter RCC Collector; based on the positive and negative controls, a cutoff of 20 was used to filter out transcript signals that registered at levels of background noise.

Statistical Interpretation of NanoString nCounter Assay:

Statistical interpretation and data visualization (heat map) was conducted using Multi Experiment Viewer (MeV) from The Institute for Genomic Research (TIGR) (Saeed et al., Methods Enzymol 411:134-193, 2006; and Saeed et al., Biotechniques 34:374-378, 2003). Using the normalized data that was exported from nCounter RCC Collector, an average of mRNA transcript probe values was taken for each cell line (three biological replicates each) representing controls (untreated) and drug-treated samples, a tab-delimited text file was generated in Microsoft Excel and uploaded into MeV. Genes were log-2 transformed and mean centered. Bi-dimensional, average-linkage, unsupervised hierarchical clustering analysis was applied to find the relationships between samples and genes using the Pearson correlation coefficient.

### Results

In Silico Docking of b-AP15 with the 19S Proteasome Associated Deubiquitinating Enzymes (DUBs), UCHL5 and USP14:

Given that UCHL5 and USP14 are the two established targets of b-AP15, their structures were first modeled in silico to determine the residues that are critical for their binding to b-AP15. A 3-dimensional protein structure was modeled for UCHL5, and found to contain a Cys88 residue that may be attacked by b-AP15 via a 1,4-Michael addition reaction. The additional reaction occurs at the thiol group (—SH) from Cys88 with the aldehyde from b-AP15. The nitrogroups from b-AP15 participate in electrostatic interactions with the Asn/Gln residues, and transient p-cloud interactions occur with the phenyl-substituted rings from b-AP15. His164 and carbonyl oxygen from b-AP15 have stabilizing interactions.

Next, USP14 was modeled, and similar to UCHL5, USP14 covalently binds b-AP15 via a 1,4-Michael addition reaction at the thiol group of the Cys114 residue (covalent linkage) with the aldehyde from the small molecule DUB inhibitor. The binding pocket was found to be highly mobile during molecular dynamics simulations (MDS), and b-AP15 binding was found to occur with cooperative changes in the pocket shape. b-AP15 shifts orientation preceding the covalent binding event at residue Cys114. Importantly, b-AP15 engagement blocks access of the C-terminal of ubiquitin from binding with USP14, which is visible in the X-ray structure of 2AYO (Hu et al., EMBO J 24:3747-3756, 2005). As with UCHL5, Asn/Gln interactions stabilize the nitro-substituted phenyl rings, while the His435 does not face the carbonyl in this insertion pose for b-AP15. b-AP15 can potentially insert in a 180°-rotated orientation, such that the DUB inhibitor faces the His435 residue similarly to UCHL5; however, molecular modelling and mechanics suggests that it has a covalent interaction with Cys411, resulting in the most optimal docking orientation.

Proteolytic Activity of the 20S Proteasome is not Compromised by b-AP15:

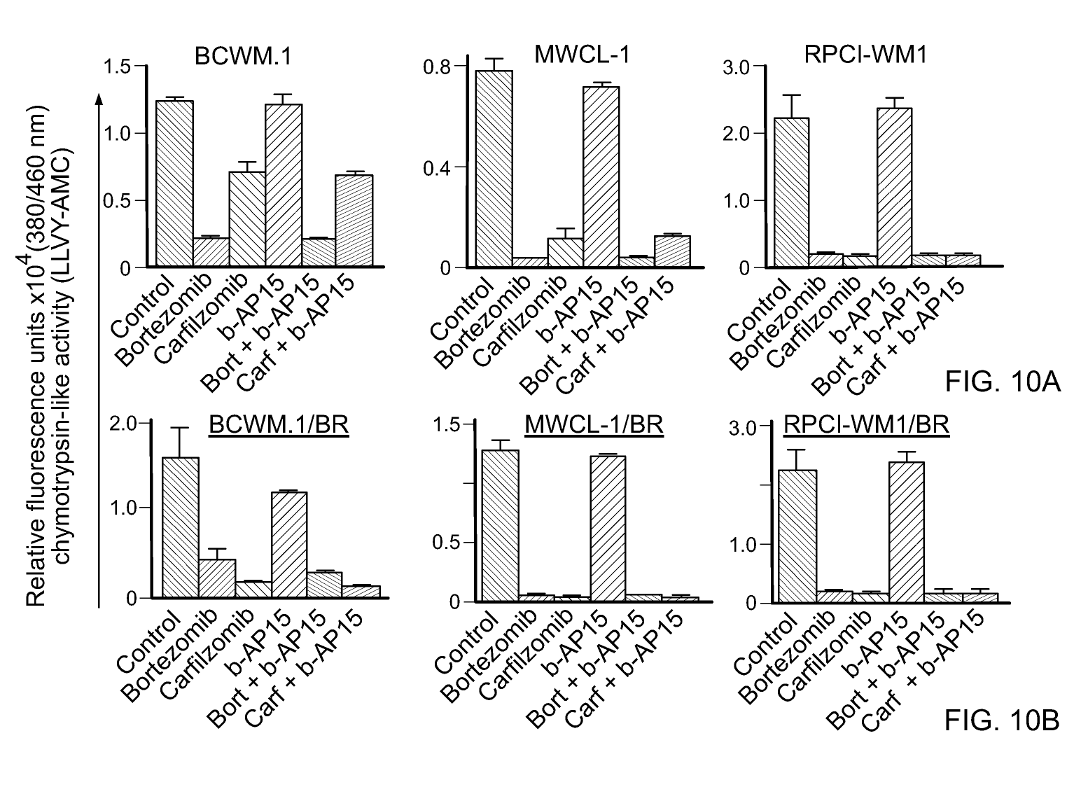

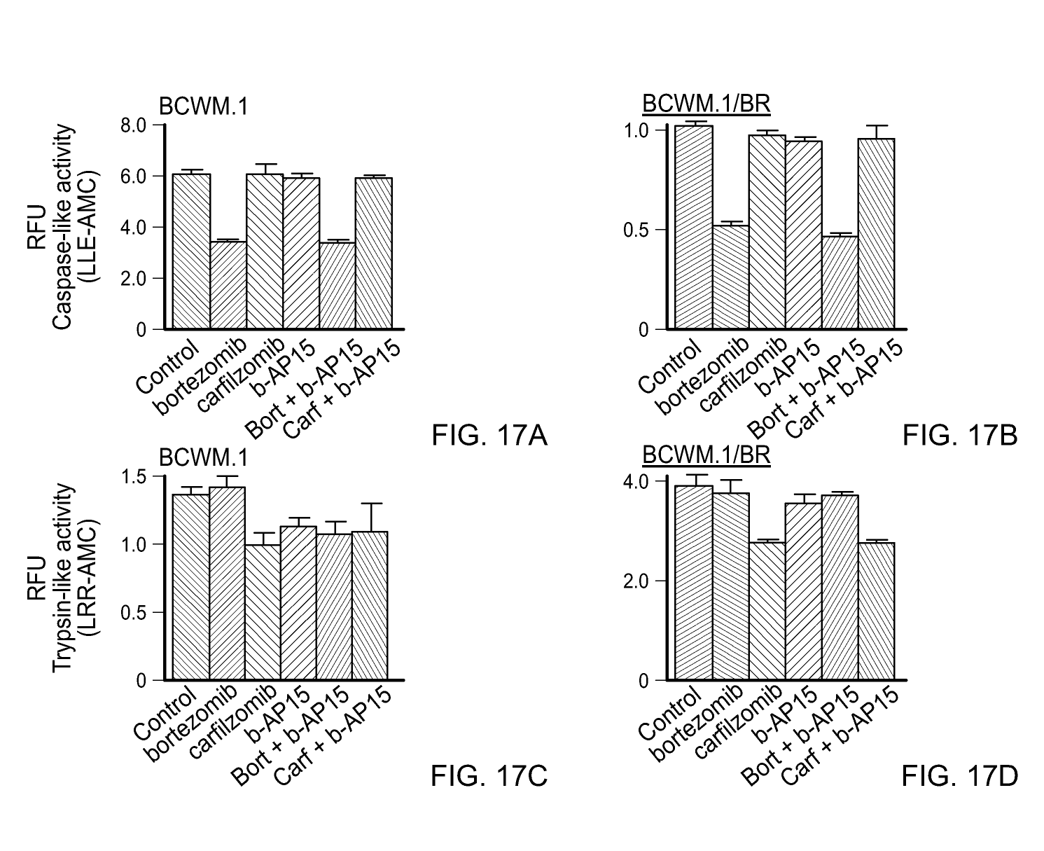

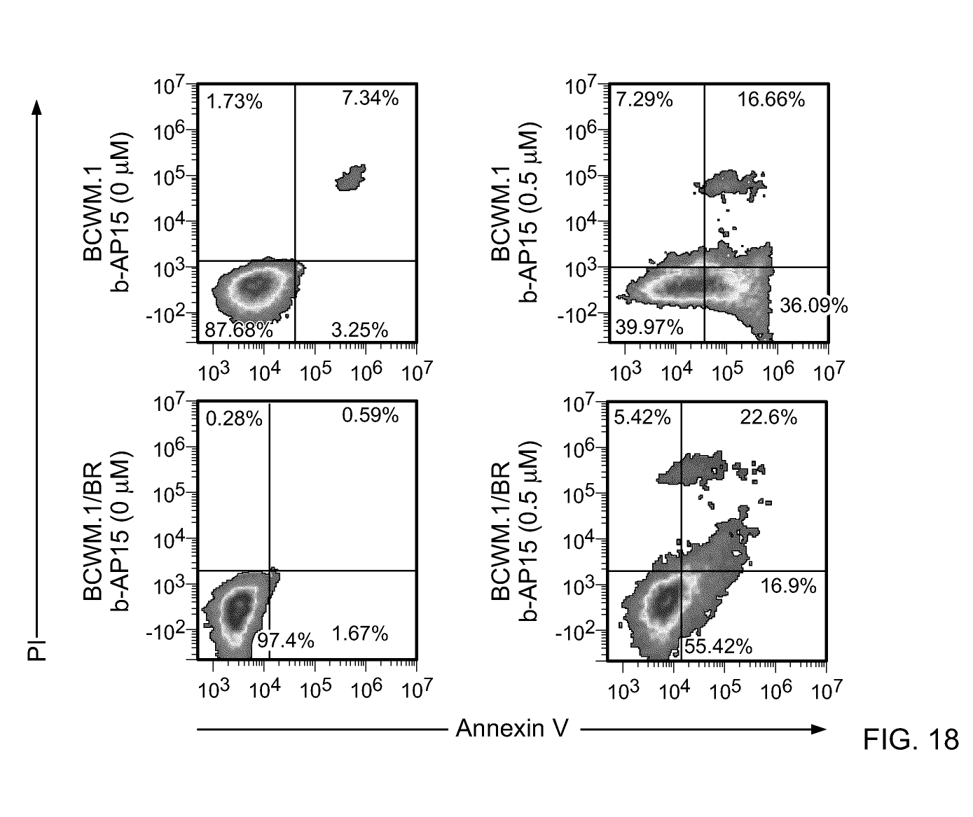

To experimentally affirm that the (19S proteasome cap) targets of b-AP15 are distinct from those of PIs such as bortezomib or carfilzomib, the enzymatic activity of the 20S proteasome b5 subunit was assessed after treatment with b-AP15±20S targeting PI (bortezomib or carfilzomib). Using a fluorogenic peptide (Suc-LLVY-AMC), which is a chymotryptic substrate, no loss of the chymotrypsin-like activity (LLVY) of the b5 subunit was observed in either bortezomib sensitive (WT) or BR WM tumor cells treated with b-AP15 (FIGS. 10A and 10B). In contrast, LLVY activity was significantly diminished in both WT and BR WM cells treated with bortezomib or carfilzomib, which served as comparators for b-AP15. Notably, addition of b-AP15 to either bortezomib or carfilzomib did not abrogate the b5 inhibitory actions of the 20S-targeting PI. No change was observed in either caspase-like (b1 subunit) or trypsin-like (b2 subunit) proteasomal activity in b-AP15-treated WM cells (FIGS. 17A-17D). This important observation affirmed that b-AP15 and established PIs target different locations (19S vs. 20S, respectively) of the proteasome, and their activity may potentially be complementary to one another. Altogether, these results demonstrate that b-AP15 does not inhibit proteasome b-catalytic function, nor does it interfere with b-catalytic activities when combined with 20S-targeting PI.

USP14 and UCHL5 are Consistently Expressed in WM Cells and their Enzymatic Inhibition with b-AP15 is Associated with an Increase in Ubiquitinated Proteins and Loss of Viability:

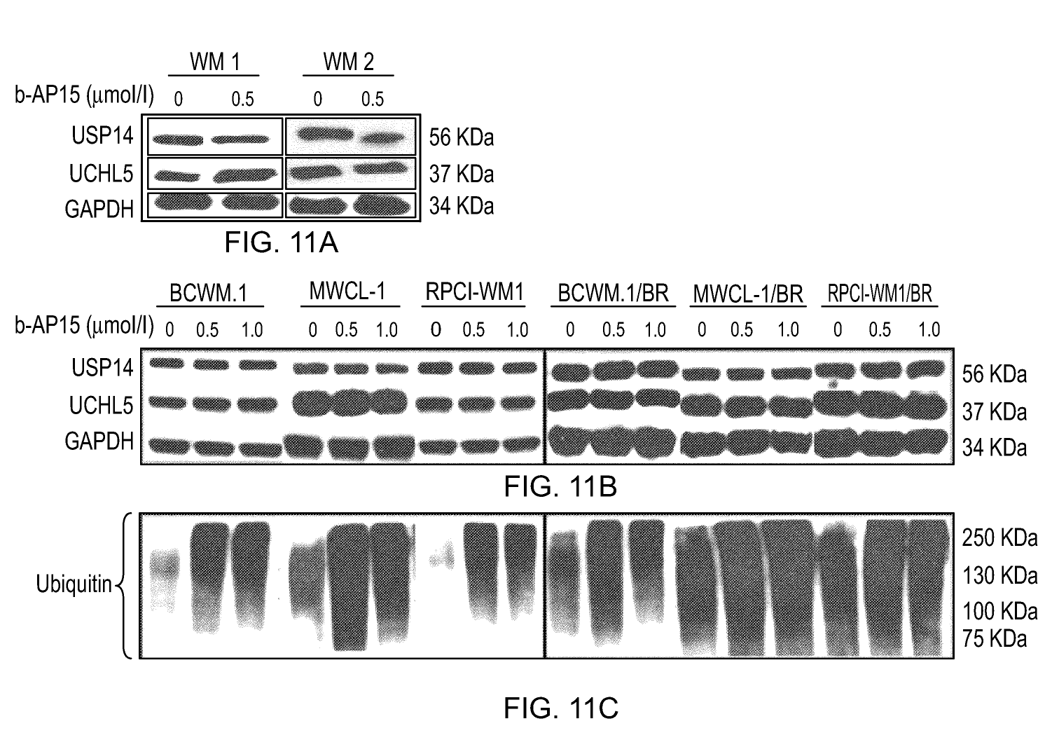

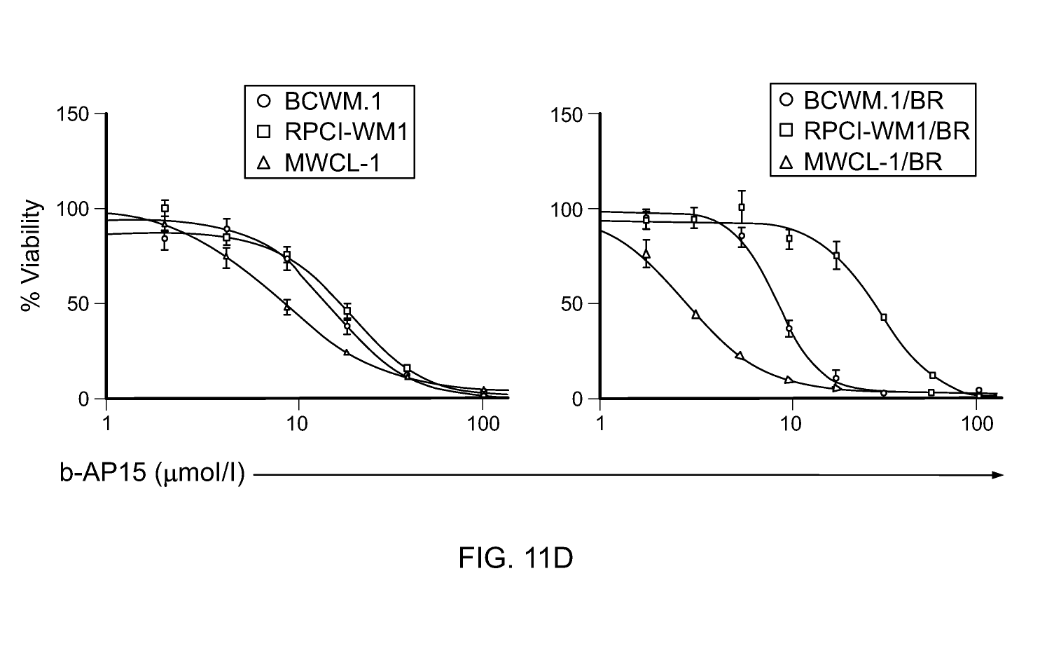

Next, studies were conducted to examine the expression of USP14 and UCHL5 proteins across WM cells. USP14 and UCHL5 protein levels were first examined in primary CD19+/CD138+ malignant WM cells from previously treated WM patients by immunoblot analysis, and notable baseline expression of the DUBs was observed, which did not change after exposure to b-AP15 (FIG. 11A). Next, this phenomenon was examined in WM cell lines (WT and BR clones), showing that USP14 and UCHL5 were consistently expressed across all WM cells, with no observable shift after b-AP15 treatment (FIG. 11B). Given that b-AP15 targets USP14/UCHL5 deubiquitinating activity, it would stand to reason that b-AP15 treatment of cells would result in build-up of ubiquitinated protein. Consistent with this, assessment of the total ubiquitinated cellular protein content revealed increasing amounts of high molecular weight polyubiquitinated conjugates in b-AP15-treated cells, in a dose-dependent manner (FIG. 11C). One of the primary mechanisms whereby PIs exert their antitumor effect is through the buildup of ubiquitinated proteins in the lumen of the endoplasmic reticulum (ER), causing ER stress beyond the threshold of what the cell can compensate for, eventually leading to apoptotic cell death (Kim et al., Apoptosis 11:5-13, 2006). To determine if the increase in polyubiquitinated conjugates in b-AP15-treated WM cells coincided with loss of tumor cell viability, a 72-hour MTS assay was conducted to assess WM cell viability following treatment with increasing concentrations of b-AP15 (0-1 μmol/l). All WM cells were noted to be exquisitely sensitive to b-AP15, with the highest sensitivity observed in MWCL-1 cells [50% inhibitory concentration (IC₅₀) 7 nmol/1], followed by BCWM.1 (IC₅₀ 13 nmol/l) and RPCI-WM1 (IC₅₀ 16 nmol/l) (FIG. 11D, left panel). The effects of b-AP15 in the corresponding BR WM cells were next assessed, revealing a loss of viability in a similar order (MWCL-1B/BR>>BCWM.1/BR>RPCI-WM1/BR) (FIG. 11D, right panel). These results affirmed USP14 and UCHL5 as valid and functional targets in WM, whose inhibition with b-AP15 results in accumulation of ubiquitinated proteins, loss of tumor cell viability, despite acquired resistance to the 20S targeting PI, bortezomib.

b-AP15 Induces Tumor-Specific Apoptosis in WM Cell Lines In Vitro and Patient-Derived WM Cells Ex Vivo:

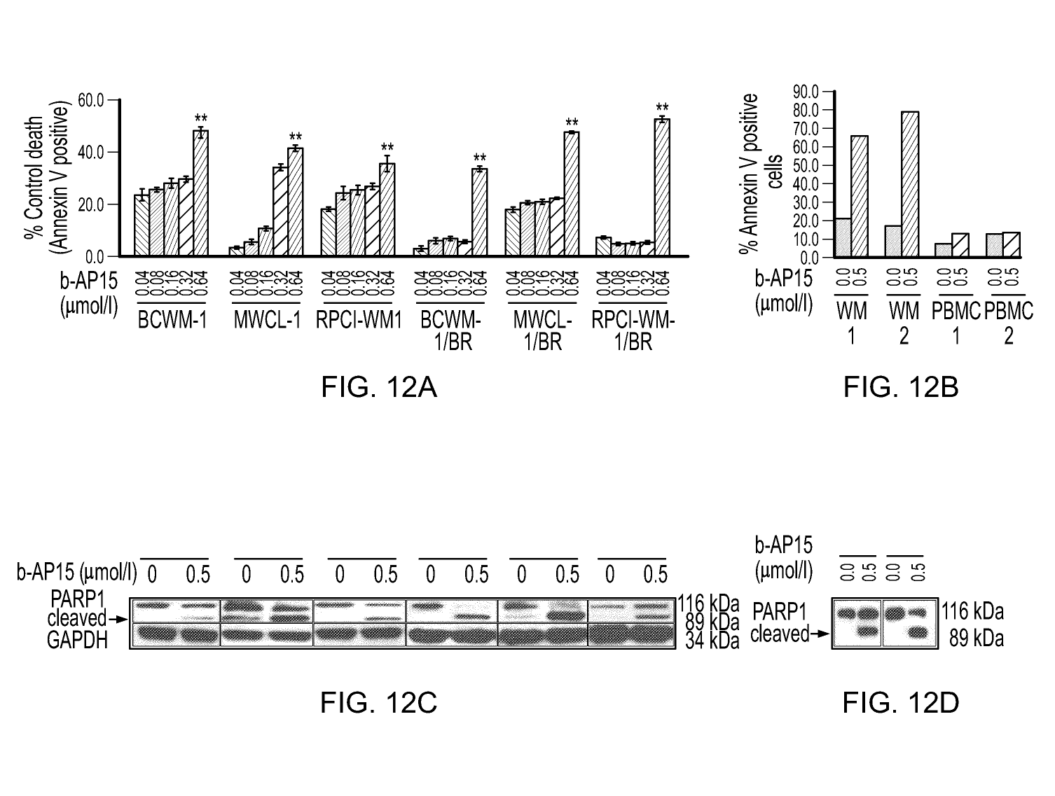

The loss of WM cell viability in the presence of b-AP15 was previously noted above, and studies were conducted to determine whether this was due to apoptotic mechanisms. All WM cell lines were treated with increasing concentrations of b-AP15 and induction of apoptosis was examined at different time points by annexin-V staining followed by flow cytometry. Among the WM models, it was observed that b-AP15 treatment caused programmed cell death as early as 6 hours, and most significantly by 12 hours in a dose-dependent manner with approximately 50% of WM cells experiencing significant apoptosis at a concentration of 0.64 μmol/l (P<0.005) (FIG. 12A, 12-h time-point shown). Heat density plots from two representative (one WT and one BR) WM models are shown in FIG. 18. Using two concentrations of b-AP15 derived from the titration (cell line-based) experiment, b-AP15-mediated apoptosis was examined in primary patient-derived WM cells as well as in PBMCs from healthy donors. Significant annexin-V positivity was noted in primary malignant cells treated with b-AP15 (0.5 μmol/l) by 12 hours (FIG. 12B), with >90% undergoing total loss of viability at a concentration of 1 μmol/l. In contrast, minimal apoptosis was observed in PBMCs cultured in b-AP15 for up to 48 hours, indicating the rapid effects of the DUB inhibitor to be tumor-cell specific. Confirmation of apoptosis in WM cell lines and patient-derived WM cells was observed by immunoblotting for PARP1 cleavage (FIGS. 12C and 12D).

Loss of Mitochondrial Transmembrane Potential is Provoked by b-AP15 in WM Cells:

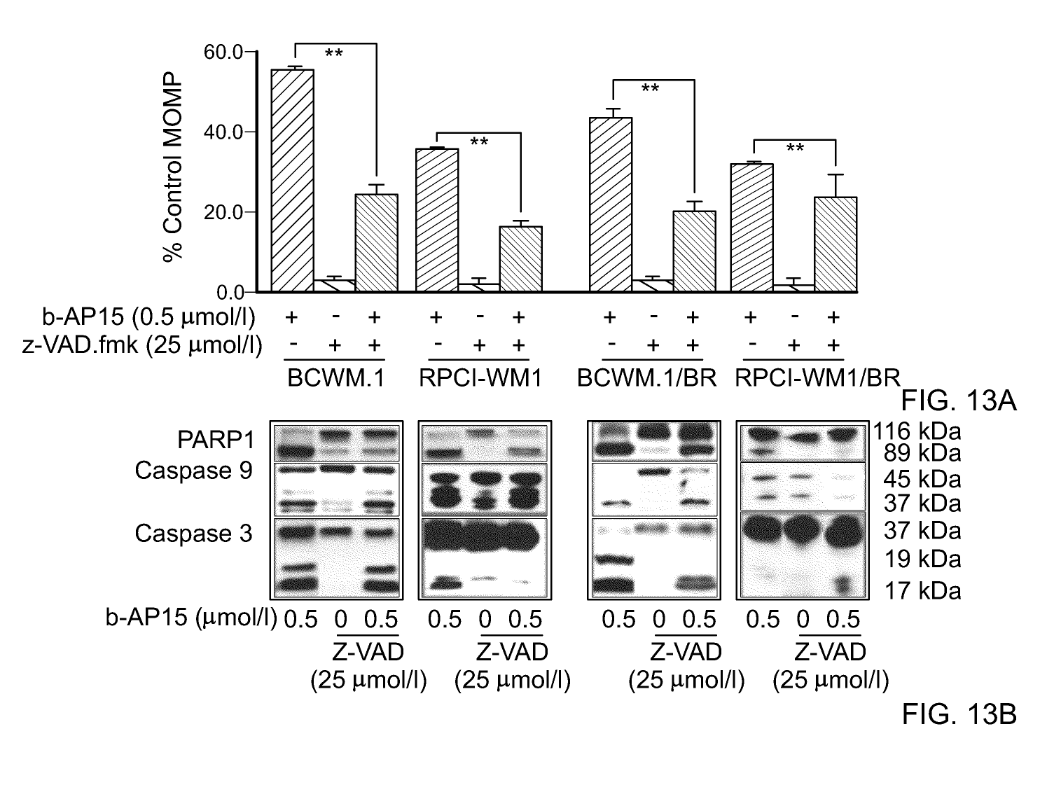

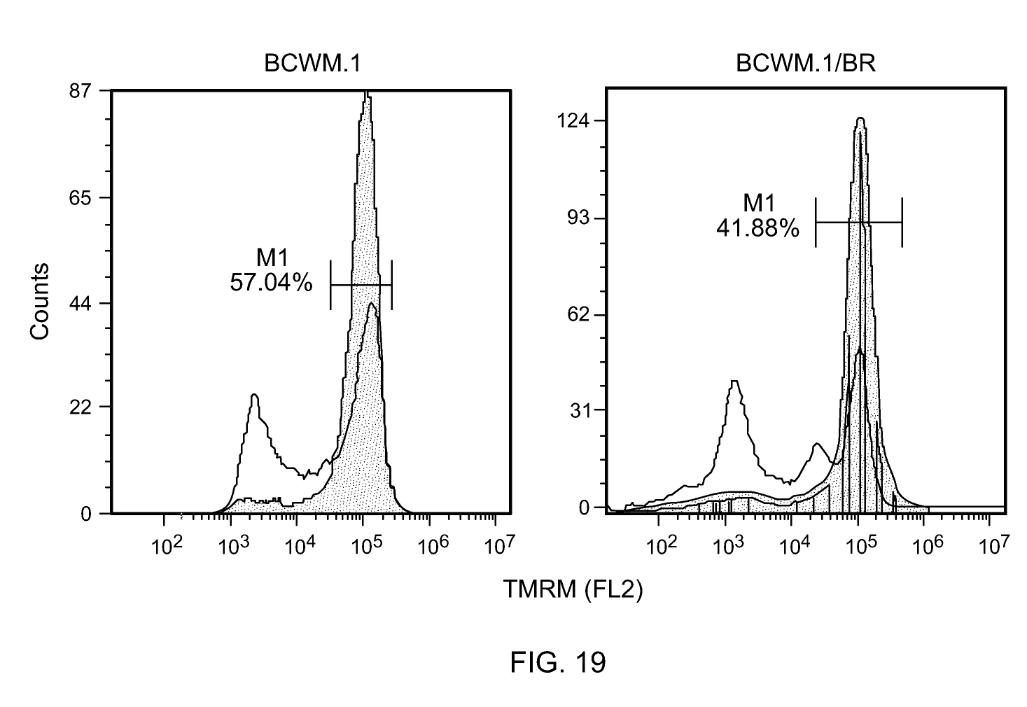

Disruption of the mitochondrial transmembrane potential (Dwm) through an increase in mitochondrial membrane permeability (MOMP) is a hallmark of death receptor-independent apoptosis and engagement of the intrinsic apoptotic cascade (Kroemer et al., Physiological Rev 87:99-163, 2007). Reports published elsewhere showed the ability of b-AP15 to induce caspase-3 cleavage; (D'Arcy et al., Nature Med 17:1636-1640, 2011), and as such, studies were conducted to determine whether the intrinsic apoptotic pathway was activated by measuring MOMP and looking for caspase-9 and -3 cleavage. MOMP was measured in relation to TMRM fluorescence in all WM cell lines and TMRM-negative cells were calculated to represent (%) MOMP. MOMP was significantly induced in b-AP15-treated WT and BR WM cells, and this coincided with PARP1 cleavage as well as cleavage of executor caspase-3 (FIGS. 13A, 13B and FIG. 19). To determine whether b-AP15-mediated toxicity was caspase-dependent, all WM cells were treated with the pan-caspase inhibitor z.VAD.fmk±b-AP15. It was observed that pre-treatment with z-VAD.fmk significantly reduced MOMP (P<0.01) in b-AP15 co-treated WT and BR WM cells. These results suggested that WM cell mitochondria are targeted by b-AP15, which disrupts the Dwm. Overall, this results in caspase-3 mediated tumor cell death, which is partially relieved by inhibition of caspase activity.

b-AP15 Modulates Genes Involved in Cellular Stress and Nuclear Factor Kappa B (NFKB1) Signaling:

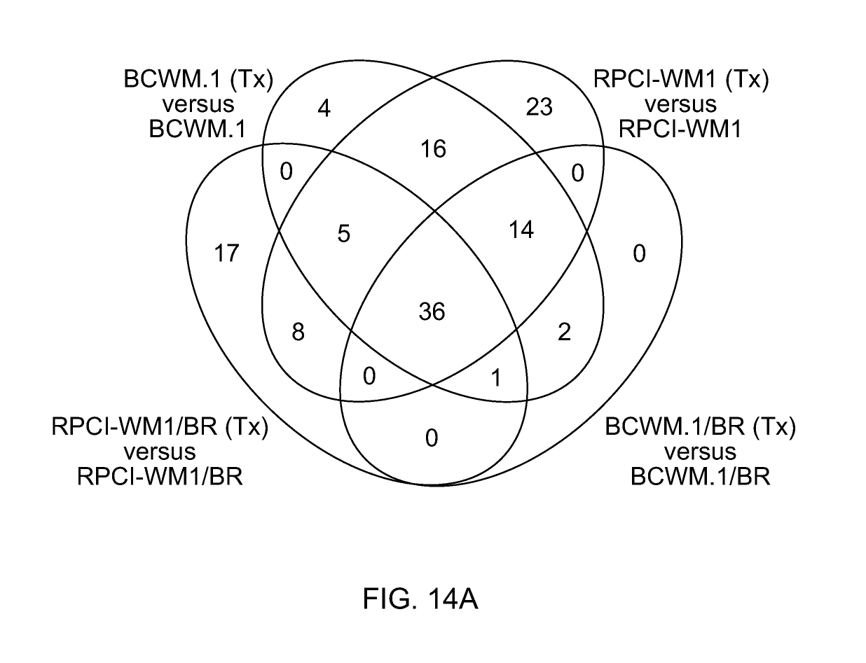

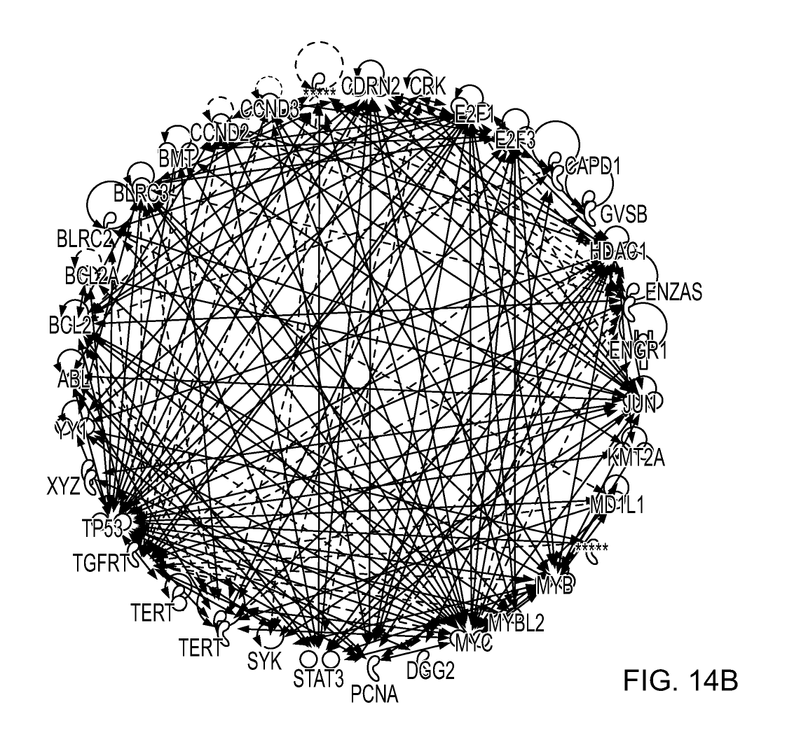

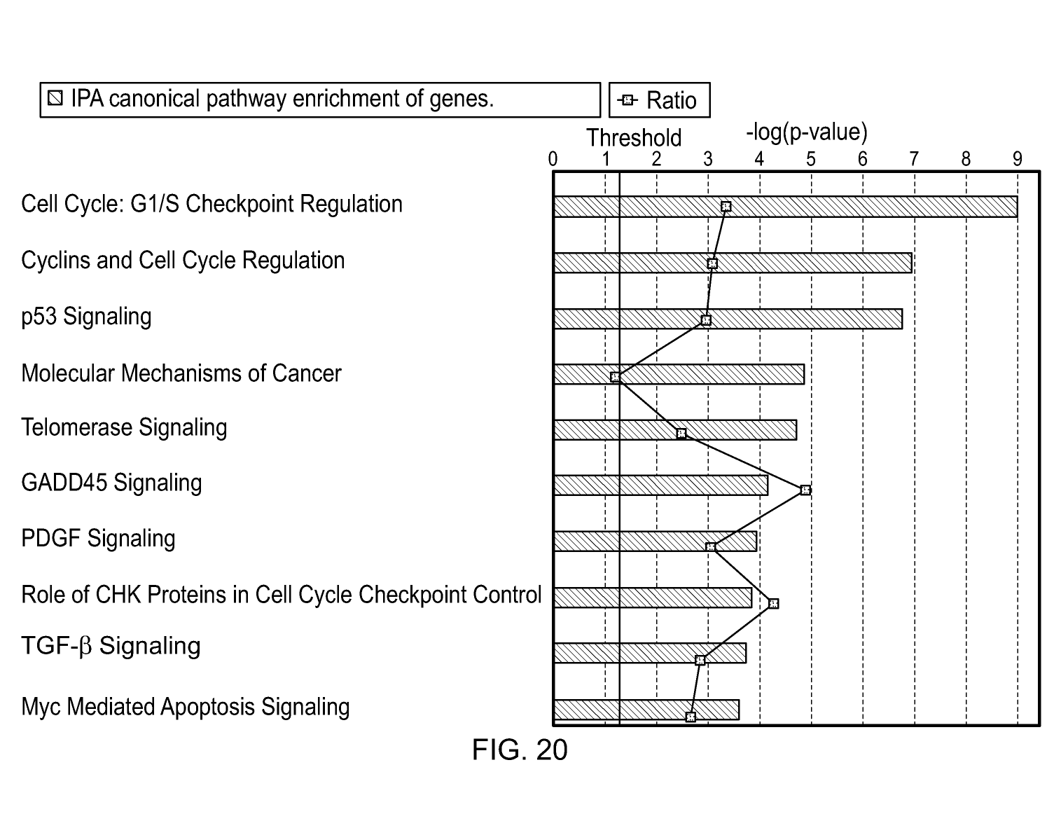

The effects of b-AP15 were examined at the transcriptional level in WM models by looking at specific cancer-related genes. The Nanostring nCounter mRNA quantification assay was used, which has a high sensitivity for direct measurement of mRNA abundance and has been demonstrated to be an equivalent alternative to quantitative real-time reverse transcription polymerase chain reaction (RT-PCR) or Open Array real-time PCR (Prokopec et al., RNA 19:51-62, 2013). BCWM.1 and RPCI-WM1 cells were treated with 50 nmol/l b-AP15, whereas their respective BR clones were treated with 100 nmol/l b-AP15 for 24 hours, followed by collection of RNA for profiling. b-AP15 treatment elicited notable changes in cancer-associated genes associated with ER/cell stress response and NFKB1 signaling mechanisms, reflected by differential expression of mRNA in each of the cell lines. To evaluate which genes were altered in the same orientation across all four cell lines, an intersect analysis was performed (FIG. 14A), identifying 36 genes that were commonly modulated, many of which have expression associated with NFKB1 signaling (TABLE 2). The relationship between the 36 genes also was explored by Ingenuity Pathway Analysis (IPA) network analysis (FIGS. 14B and 20), illustrating the interaction and relative expression of these genes. This analysis shed light on the protein interactions that remain critical for WM cell survival, irrespective of resistance to b5-targeting PI, and that can be modulated by b-AP 15 therapy.

Activation and Nuclear Translocation of RELA (p6.5) is Attenuated by b-AP15:

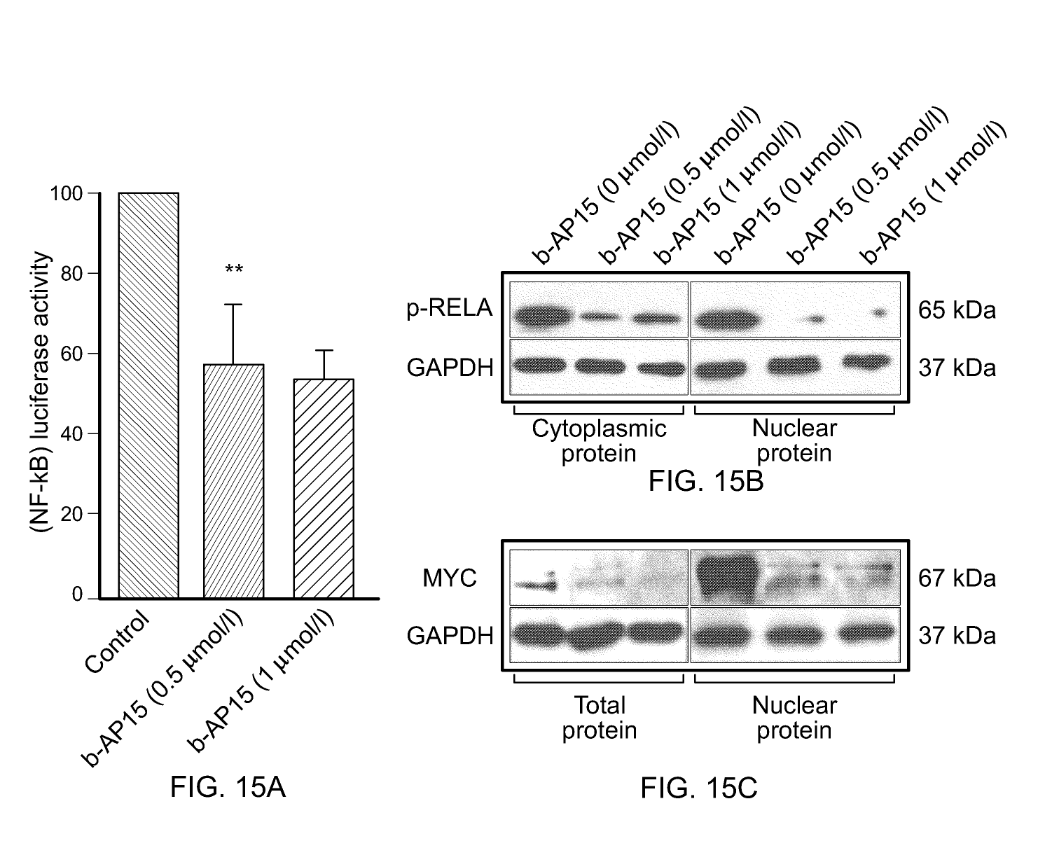

Abnormalities in the NFKB1 signaling pathway have been implicated in WM cell growth and survival (Leleu et al., Blood 111:5068-5077, 2008; Braggio et al., Cancer Res 69: 3579-3588, 2009; and Ansell et al., Blood Cancer J4:e183, 2014). Moreover, nearly all WM cases (˜97%) carry a mutant MYD88 gene (MYD88L265P), which hyperactivates NFKB1 by constitutively associating itself with IRAK4 and TRAF6 (Treon et al., New Engl J Med, 367:826-833, 2012). With this in mind, the impact of b-AP15 on mutant MYD88-directed activation of NFKB1 was interrogated through a NFKB1 luciferase reporter assay in MYD88L265P expressing HEK293T cells (Ansell et al., 2014, supra). Transfected 293T cells were treated with b-AP15 for 24 hours, and untreated cells were used as a control for comparison. As expected, treatment with b-AP15 (0.5 μmol/l) significantly reduced NFKB1 luciferase activity, indicating a decrease in NFKB1 gene activation (P<0.004, FIG. 15A). Further, a marked reduction in nuclear RELA (p-p65) availability was confirmed directly at the protein level in b-AP15 treated WM cells (FIG. 15B; results from one representative model shown). Indeed, these observations were supported by NanoString data analysis, which showed downregulation of NFKB1 target genes (TABLE 2). This was experimentally confirmed by examining the NFKB1 target, MYC, in BCWM.1 cells, which showed reduced total and nuclear expression after treatment with b-AP15. Altogether, these data revealed that b-AP15 decreases the nuclear translocation and activation potential of NFKB1 and target genes, such as MYC, in MYD88L265P WM cells.

b-AP15 Causes a Shift in the ER and Cell-Stress Response Proteins in WM Cells:

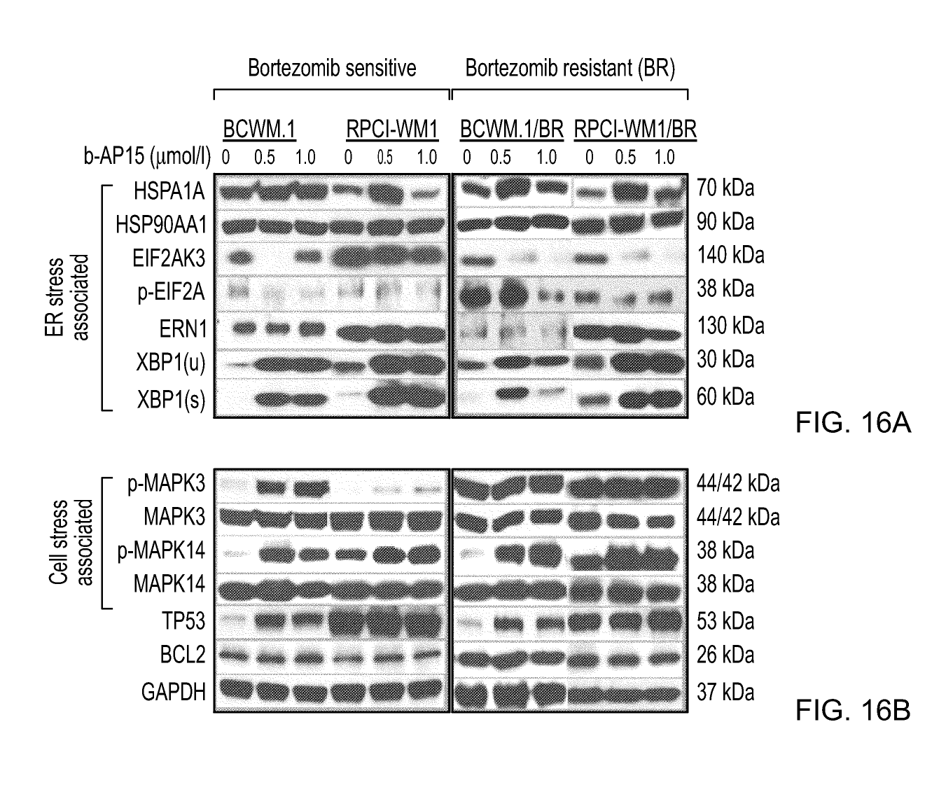

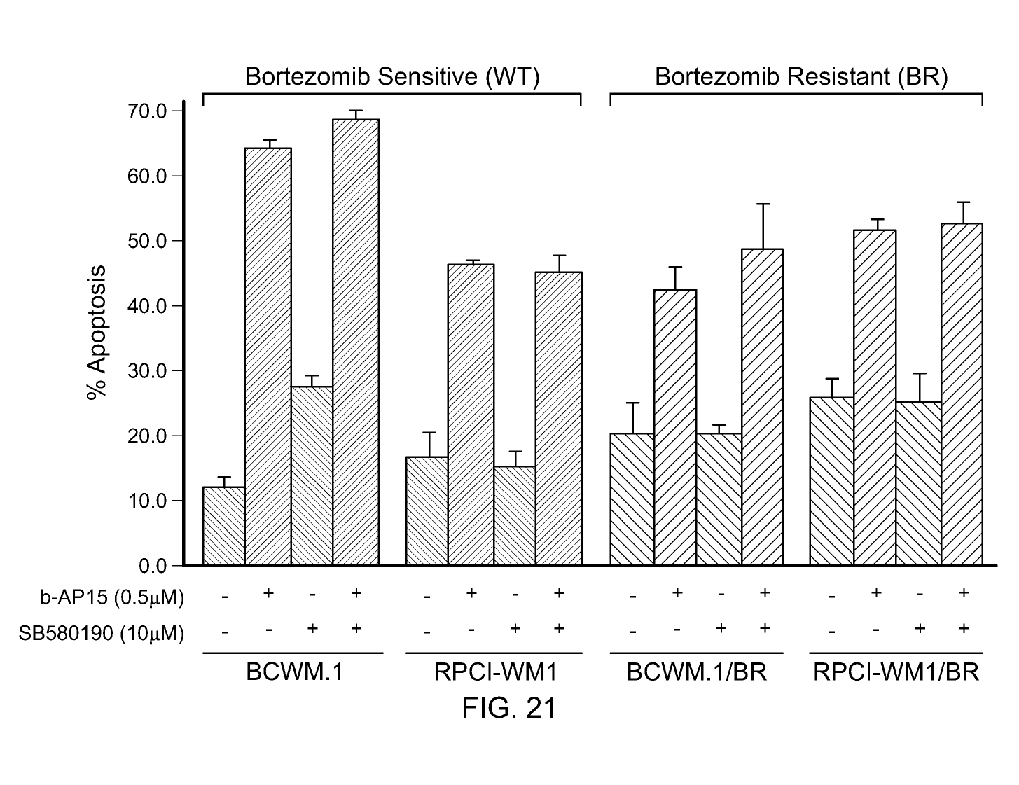

b-AP15 has a clear effect on ER stress, unfolded protein response (UPR) and cell stress-associated elements (Brnjic et al., Antioxidants Redox Signaling 21:2271-2285, 2014; and Tian et al., Blood 123:706-716, 2014). In line with observations described elsewhere (D'Arcy et al., supra; Brnjic et al., supra), it was noted that ER stress machinery, such as HSPA1A, was consistently present in both WT and BR cell lines, and notably more so after b-AP15 treatment (FIG. 16A). In addition, XBP1 and its spliced active form, XBP1s, which are primary effectors of the UPR (Ron and Walter, Nature Reviews: Mol Cell Biol, 8:519-529, 2007), were found to be significantly induced by b-AP15 across all cell lines. Another UPR effector, EIF2AK3 (PERK) was notably present in all untreated WM cells. In BR models, b-AP15 decreased EIF2AK3 levels; however, this effect was not concordantly seen in WT cell lines. Expression of the EIF2AK3 target, p-EIF2a, did not appear to change following b-AP15 treatment. In addition to ER stress, it has been noted that b-AP15 activates cell stress-related kinases, as evidenced by modulation of MAPK proteins and downstream activation of their target transcription factors (FIG. 16B and TABLE 2, Jun/Fos upregulation; see, also, Brnjic et al., supra). An increase in phosphorylated-MAPK3/MAPK1 (ERK1/2) also was observed in WT cell lines after b-AP15 treatment; however, this was not observed in BR models. In addition, there was a significant increase of phosphorylated MAPK14 (p38) protein after b-AP15 (6-hour treatment). MAPK14 is galvanized in response to DNA damage or cell stress and can act as either a compensatory prosurvival protein or facilitate cell death, depending on the cellular context (Wagner and Nebreda, Nature Reviews: Cancer 9:537-549, 2009). To determine its significance, the MAPK14 inhibitors (SB202190 or SB580190) were used alone and in combination with b-AP15. Although greater loss of tumor cell viability was observed when a MAPK14 inhibitor was combined with a low concentration of b-AP15 (100 nmol/l), this effect was not observed with higher concentrations of b-AP15+ MAPK14 inhibitor (FIG. 21). Lastly, studies were conducted to assess the protein levels of TP53 and BCL2, whose dysregulated activity is implicated in bortezomib-resistance (D'Arcy et al., supra; Paulus et al., 2014). No change in BCL2 was observed, but a marginal increase in TP53 across b-AP15-treated WT and BR WM cells was noted. This is in line with reports described elsewhere, which showed similar findings and demonstrated that although TP53 is induced, b-AP15 anti-tumor activity is not TP53-dependent (D'Arcy et al., supra).

TABLE 1
Sensitivity of WM cell lines and BR subclones to bortezomib

| Bortezomib sensitive WM | Bortezomib resistant (BR) |
|---|---|
| cell lines (IC₅₀ nM) | WM subclones (IC₅₀ nM) |
| BCWM.1 (11.4) | BCWM.1/BR (720.0) |
| MWCL-1 (16.6) | MWCL-1/BR (65.2) |
| RPCI-WM1 (27.0) | RPCI-WM1/BR (125.0) |

MTS assay performed to derive IC₅₀ values.

TABLE 2
Genes commonly altered in bortezomib-sensitive
and BR WM cells with b-AP15

| | Average fold |
|---|---|
| Gene | change |
| IL8 | 14.77069258 |
| SERPINE1 | 5.733784303 |
| JUN | 5.625177091 |
| FOS | 5.569160897 |
| EGR1 | 2.269780634 |
| BIRC5 | −2.209428118 |
| SPI1 | −2.52161554 |
| CDK4 | −2.590178275 |
| MYBL2 | −2.793659211 |
| AKT2 | −2.812484515 |
| XRCC5 | −2.834353025 |
| HDAC1 | −2.886544075 |
| CCND2 | −2.999961091 |
| RAD54L | −3.028989277 |
| XPC | −3.044888976 |
| BMI1 | −3.090253662 |
| PCNA | −3.166406233 |
| TFRC | −3.192086864 |
| HRAS | −3.27664425 |
| MYC | −3.286507033 |
| CDC25B | −3.62932228 |
| E2F1 | −3.634275978 |
| CASP2 | −3.67883593 |
| TUBB | −3.724226978 |
| SYK | −3.839839192 |
| CSK | −3.871268989 |
| TYMS | −3.965938423 |
| CCNE1 | −4.19313578 |
| MSH6 | −4.242372803 |
| TFDP1 | −4.261070292 |
| MSH2 | −4.366742879 |
| ETV6 | −4.64251861 |
| CDC2 | −4.667621711 |
| CCNA2 | −5.840799265 |
| TOP2A | −6.321475694 |
| MYB | −9.646723441 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating blood cell cancer in a mammal, wherein said method comprises:
(a) administering bortezomib to said mammal,
(b) detecting the presence of blood cancer cells within said mammal that have an elevated level of PSMB9 expression, and
(c) administering carfilzomib to said mammal.

2. The method of claim 1, wherein said mammal is a human.

3. The method of claim 1, wherein the presence of said blood cancer cells is detected using a quantitative polymerase chain reaction assay to measure PSMB9 mRNA levels.

4. The method of claim 1, wherein the presence of said blood cancer cells is detected using a polypeptide detection assay for detecting β1i polypeptide levels.

5. The method of claim 1, wherein said blood cancer cells are lymphoma cells.

6. The method of claim 5, wherein the lymphoma cells are mantle cell lymphoma (MCL) cells, Waldenströms macroglobulinemia (WM) cells, or diffuse large B-cell lymphoma (DLBCL) cells.

7. The method of claim 1, wherein said blood cancer cells are myeloma cells.

8. A method for treating blood cell cancer in a mammal, wherein said method comprises:
(a) administering bortezomib to said mammal,
(b) detecting the presence of blood cancer cells within said mammal that have an elevated level of PSMB9 expression, and
(c) administering VLX1570 to said mammal.

9. The method of claim 8, wherein said mammal is a human.

10. The method of claim 8, wherein the presence of said blood cancer cells is detected using a quantitative polymerase chain reaction assay to measure PSMB9 mRNA levels.

11. The method of claim 8, wherein the presence of said blood cancer cells is detected using a polypeptide detection assay for detecting β1i polypeptide levels.

12. The method of claim 8, wherein said blood cancer cells are lymphoma cells.

13. The method of claim 12, wherein the lymphoma cells are MCL cells, WM cells, or DLBCL cells.

14. The method of claim 8, wherein said blood cancer cells are myeloma cells.
